(19) United States
(12) Patent Application Publication
(10) Pub. No.: US 2004/0067526 A1
(43) Pub. Date: Apr. 8, 2004

(54) Methods for diagnosing and guiding treatment of bone turnover disease

(76) Inventor: Thomas L. Cantor, El Cajon, CA (US)

Correspondence Address: David Devernoe, Morrison & Foerster LLP, Suite 500, 3811 Valley Centre Drive, San Diego, CA, 92130-2332, US

(21) Appl. No.: 10/265,276

(22) Filed: Oct. 3, 2002

(51) Int. Cl. G01N 33/53

(52) U.S. Cl. 435/7.1

(57) ABSTRACT

The present invention relates to parathyroid hormone (PTH) level determinations, in particular the determination total PTH, PTH agonist, PTH antagonist levels and comparisons between these levels. These calculated levels may be adjusted and are useful for determining a person's bone turnover rate, including determining the risk of a person for a bone turnover-related disease and guiding treatment therefor.

### CROSS-REFERENCE TO RELATED APPLICATIONS

This application is related to U.S. patent application Ser. No. 09/231,422, filed Jan. 14, 1999; U.S. patent application Ser. No. 09/344,639, filed Jun. 26, 1999; U.S. patent application Ser. No. 10/002,818, filed Nov. 2, 2001; and U.S. patent application Ser. No. 10/215,770, filed Aug. 9, 2002, all of which are incorporated herein by reference.

### TECHNICAL FIELD

The present invention relates to parathyroid hormone (PTH) level determinations, in particular the determination total PTH, PTH agonist, PTH antagonist levels and comparisons between these levels. These calculated levels may be adjusted and are useful for determining the risk of a person for an adynamic low bone turnover disease, high bone turnover disease and guiding treatment therefor.

### BACKGROUND OF THE INVENTION

Calcium plays an indispensable role in cell permeability, the formation of bones and teeth, blood coagulation, transmission of nerve impulse, and normal muscle contraction. The concentration of calcium ions in the blood is, along with calcitriol and calcitonin, regulated mainly by parathyroid hormone (PTH). Extracellular calcium levels are directly affected by PTH through calcium uptake in kidney tubule cells and calcium transport to or from bone. Although calcium intake and excretion may vary, PTH serves through feedback mechanism to maintain a steady concentration of calcium in cells and surrounding fluids. When serum calcium lowers, the parathyroid glands secrete PTH, affecting the release of stored calcium. When serum calcium increases, stored calcium release is retarded through lowered secretions of PTH.

Osteoporosis is the most common form of metabolic bone disease and may be considered the symptomatic, fracture stage of bone loss (osteopenia). Although osteoporosis may occur secondary to a number of underlying diseases, 90% of all cases appear to be idiopathic. Postmenopausal women are particularly at risk for idiopathic osteoporosis (postmenopausal or Type I osteoporosis). Another high risk group for idiopathic osteoporosis is the elderly of either sex (senile or Type II osteoporosis). Osteoporosis has also been related to corticosteroid use, immobilization or extended bed rest, alcoholism, diabetes, gonadotoxic chemotherapy, hyperprolactinemia, anorexia nervosa, primary and secondary amenorrhea, and oophorectomy.

The complete or whole form of human PTH, (hPTH), is a unique 84 amino acid peptide (SEQ ID NO: 1), as is shown in FIG. 1. Researchers have found that this peptide has an anabolic effect on bone that involves a domain for protein kinase C activation (amino acid residues 28 to 34) as well as a domain for adenylate cyclase activation (amino acid residues 1 to 7). However, various catabolic forms of clipped or fragmented PTH peptides also are found in circulation, most likely formed by intraglandular or peripheral metabolism. For example, hPTH can be cleaved between amino acids 34 and 35 to produce a (1-34) PTH N-terminal fragment and a (35-84) PTH C-terminal fragment. Likewise, clipping can occur between either amino acids 36 and 37 or 37 and 38. Recently, a large PTH fragment referred to as “non-(1-84) PTH” has been disclosed which is clipped closer to the N-terminal end of PTH. (See LePage, R., et al., “A non-(1-84) circulating parathyroid hormone (PTH) fragment interferes significantly with intact PTH commercial assay measurements in uremic samples.” Clin. Chem. (1998); 44: 805-810.)

The cleaved fragments of PTH vary in both biological activity and metabolic clearance rate from the circulation. For example, the N-terminal human PTH₁₋₃₄ (hPTH₁₋₃₄) fragment has PTH agonist properties, but is rapidly removed from circulation. A daily subcutaneous administration of hPTH to patients with idiopathic osteoporosis has been shown to substantially increase their iliac trebecular bone volume. (See Podbesek et al., Endocrinology, 112:1000-1006 (1983)).

PTH plays a role in the course of disease in a patient with chronic renal failure. Renal osteodystrophy (RO) is a complex skeletal disease comprising osteitis fibrosa cystica (caused by PTH excess), osteomalacia, resulting in unmineralized bone matrix (caused by vitamin D deficiency), extraskeletal calcification/ossification (caused by abnormal calcium and phosphorus metabolism), and adynamic bone disease (contributed to by PTH suppression). Chronic renal failure patients can develop RO. Failing kidneys increase serum phosphorus (hyperphosphoremia) and decrease 1,25-dihydroxyvitamin D (1,25-D) production by the kidney. The former results in secondary hyperparathyroidism from decreased gastrointestinal calcium absorption and osteitis fibrosa cystica from increased PTH in response to an increase in serum phosphorus. The later causes hypocalcemia and osteomalacia. With the onset of secondary hyperparathyroidism, the parathyroid gland becomes less responsive to its hormonal regulators because of decreased expression of its calcium and vitamin D receptors. Serum calcium drops. RO can lead to digital gangrene, bone pain, bone fractures, and muscle weakness.

This invention is based on the concept that RO is the result of two basic maladies of bone turnover. One underlying malady of RO is adynamic low bone turnover disease and the other underlying malady of RO is high bone turnover disease. It is known that bone is healthy and in its strongest, non degenerative state when it is turning over or remodeling at an optimal rate, so called, “normal bone turnover”. A state defined by a bone turnover (or remodeling) rate that is too low, is termed adynamic low bone turnover disease; conversely, a state defined by a bone turnover rate that is too high is termed high bone turnover disease. The present invention is based, in part, on the premise that there are two hormones (both secreted by the parathyroid gland) that are antagonists which exert control over the rate of bone turnover. CAP (cyclase activating PTH or PTH agonist) or 1-84 PTH operating through the PTH/PTHrp receptor accelerates bone turnover, and CIP (cyclase inactive PTH or PTH antagonist), frequently comprised of 7-84 PTH, operates through a C terminal PTH receptor and decelerates bone turnover.

Typically, a bone disease patient must receive continuous therapy, for life. These patients are in need of accurate diagnosis and treatment monitoring. Because of the trauma and inconvenience of surgically invasive bone biopsies in order to determine the bone turnover status, a typical bone disease patient never undergoes a single bone biopsy. Frequently, if a bone disease patient is subject to a bone biopsy on one occasion, it is a rare occurrence if the same patient has a second bone biopsy. Yet, in many cases a bone disease patient requires monthly determinations of bone turnover status in order that bone affecting therapy might be guided. The number of patients afflicted by bone disease currently exceeds 10 million in the United States alone. Therefore, there exists a strong need in the art for a non-invasive method to both initially diagnose bone turnover disease and to frequently monitor bone turnover status in a non traumatic and accurate manner during on going therapy. The present invention addresses this and other related needs in the art.

### SUMMARY OF THE INVENTION

In one embodiment a method is provided for assessing a person's bone turnover rate comprising: a) obtaining a sample from a person to be tested; b) determining the level of a parathyroid hormone (PTH) agonist and a PTH antagonist in the sample; c) obtaining a ratio of the PTH agonist versus the PTH antagonist for the person; and d) comparing the ratio obtained in step c) to a list of probabilities for predicting adynamic low bone turnover disease expressed as a percentage for accurate prediction of an adynamic low bone turnover disease, the probabilities being in a relationship to PTH agonist/antagonist ratios based on a dialysis patient population, wherein the population has a clinically significant risk of an adynamic low bone turnover disease below a normal PTH agonist/antagonist ratio range within a target ratio range of between about 1.17 to about 3.15, and wherein the person is determined as having an adynamic low bone turnover disease if the ratio of step c) is below the normal range within the target ratio range.

In one aspect, the PTH antagonist level is determined by determining a total PTH level and determining a PTH agonist level followed by subtracting the PTH agonist level from the total PTH level.

In a further aspect, the PTH agonist comprises a contiguous portion of human PTH having an amino acid sequence set forth in SEQ ID NO: 1 (PTH₁₋₈₄), and the PTH agonist has the following characteristics: the N-terminal amino acid residue of the PTH agonist starts at position 1 of the PTH₁₋₈₄; and the C-terminal amino acid residue of the PTH agonist ends at any position spanning position 34 through position 84 of the PTH₁₋₈₄. In another aspect, the PTH antagonist comprises a contiguous portion of human PTH having an amino acid sequence set forth in SEQ ID NO: 1 (PTH₁₋₈₄), and the PTH antagonist has the following characteristics: the N-terminal amino acid residue of the PTH antagonist starts at any position spanning position 2 through position 33 of the PTH₁₋₈₄; the C-terminal amino acid residue of the PTH antagonist ends at any position spanning position 35 through position 84 of the PTH₁₋₈₄; and the PTH antagonist has a minimal length of three amino acid residues.

Frequently, the parathyroid hormone agonist level is determined using an antibody that distinguishes PTH agonist from PTH antagonist. And, the parathyroid hormone antagonist level is frequently determined using an antibody that distinguishes PTH antagonist from PTH agonist.

In another embodiment a method is provided for assessing a person's bone turnover rate comprising: a) obtaining a sample from a person to be tested; b) determining and comparing a total PTH level by two assays to generate a total PTH bias factor, the assays comprising (1) a Scantibodies Laboratory Total Intact PTH Assay or a Scantibodies Laboratory Intact PTH Assay, or a combination thereof, and (2) a non-Scantibodies Laboratory intact PTH assay; c) determining and comparing a PTH agonist level by two PTH assays to generate a PTH agonist bias factor, the assays comprising (1) a Scantibodies Laboratory Whole PTH Assay or a Scantibodies Laboratory CAP Assay, or a combination thereof, and (2) a non-Scantibodies Laboratory 3^{rd} generation PTH assay; d) adjusting the total PTH level determined by the non-Scantibodies Laboratory intact PTH assay, whereby the total PTH bias factor is multiplied by the total PTH level determined by the non-Scantibodies Laboratory intact PTH assay to obtain an adjusted total PTH level; e) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level; f) obtaining an adjusted PTH antagonist level by subtracting the adjusted PTH agonist level from the adjusted total PTH level; g) obtaining an adjusted ratio of the adjusted PTH agonist versus the adjusted PTH antagonist; and h) comparing the adjusted ratio to a list of probabilities expressed as a percentage for an adynamic low bone turnover disease, the probabilities being in a relationship to PTH agonist/antagonist ratios based on a dialysis patient population, wherein the population has a clinically significant risk of an adynamic low bone turnover disease below a normal PTH agonist/antagonist ratio range within a target ratio range of between about 1.17 to about 3.15, and wherein the person is determined as having an adynamic low bone turnover disease if the adjusted ratio of step g) is below the normal range within the target ratio range.

In a particular related aspect, the total PTH bias factor is obtained by dividing the total PTH value obtained through the practice of the step b)(1) by the corresponding total PTH value obtained through the practice of the assay of step b)(2); and wherein the PTH agonist bias factor is obtained by dividing the PTH agonist value obtained through the practice of the assay of step c)(1) by the corresponding PTH agonist value obtained through the practice of step c)(2). And, frequently the non-Scantibodies Laboratory 3rd generation PTH assay or the non-Scantibodies PTH assay is the same or different assay between steps b) and c).

In another embodiment, a method is provided for assessing a person's bone turnover rate comprising: a) obtaining a sample from a person to be tested; b) determining the level of a parathyroid hormone (PTH) agonist; and c) comparing the PTH agonist level to a list of probabilities for predicting adynamic low bone turnover disease expressed as a percentage for accurate prediction of an adynamic low bone turnover disease, the probabilities being in a relationship to PTH agonist levels based on a dialysis patient population, wherein the population has a clinically significant risk of an adynamic low bone turnover disease below a normal PTH agonist range within a target PTH agonist range of between about 83 pgm/ml to about 412 pgm/ml, and wherein the person is determined as having an adynamic low bone turnover disease if the PTH agonist level is below the normal range within the target range. Frequently, the person has a clinically significant risk of an adynamic low bone turnover disease at a PTH antagonist level of below about 127 pgm/ml.

In yet another embodiment a method is provided for assessing a person's bone turnover rate comprising: a) obtaining a sample from a person to be tested; b) determining the level of a parathyroid hormone (PTH) antagonist; and c) comparing the PTH antagonist level to a list of probabilities for predicting adynamic low bone turnover disease expressed as a percentage for accurate prediction of an adynamic low bone turnover disease, the probabilities being in a relationship to PTH antagonist levels based on a dialysis patient population, wherein the population has a clinically significant risk of an adynamic low bone turnover disease above a normal PTH antagonist range within a target PTH antagonist range of between about 14 pgm/ml to about 91 pgm/ml, and wherein the person is determined as having an adynamic low bone turnover disease if the PTH antagonist level is above the normal range within the target range. Frequently, the person has a clinically significant risk of an adynamic low bone turnover disease at a PTH antagonist level of above about 63 pgm/ml.

In a particular set of embodiments, methods are provided for guiding therapy based on PTH agonist/antagonist ratios (adjusted or unadjusted), PTH agonist levels (adjusted or unadjusted), and PTH antagonist levels (adjusted or unadjusted). In a particular embodiment, a method is provided for guiding therapy for persons suspected of having an adynamic low bone turnover disease comprising determining a PTH agonist/antagonist ratio, whether it is adjusted or unadjusted, and determining therapy based thereon, wherein: a) at a ratio below the normal ratio range, therapy to increase the bone turnover rate in the person is started or increased, or therapy to decrease the bone turnover rate in the person is halted or decreased; b) at a ratio above the normal ratio range, therapy to decrease the bone turnover rate in the person is started or increased, or therapy to increase the bone turnover rate in the person is halted or decreased; and c) at a ratio within the normal ratio range, no bone turnover-related therapy is begun or altered.

In another embodiment a method is provided for guiding therapy for a person suspected of having an adynamic low bone turnover disease comprising determining the PTH agonist level, adjusted or unadjusted, and determining therapy based thereon, wherein: a) at a PTH agonist level below the normal range, therapy to increase the bone turnover rate in the person is started or increased, or therapy to decrease the bone turnover rate in the person is halted or decreased; b) at a PTH agonist level above the normal range, therapy to decrease the bone turnover rate in the person is started or increased, or therapy to increase the bone turnover rate in the person is halted or decreased; and c) at a PTH agonist level within the normal range, no bone turnover-related therapy is begun or altered.

In another embodiment a method is provided for guiding therapy a person suspected of having an adynamic low bone turnover disease comprising determining the PTH antagonist level, adjusted or unadjusted, and determining therapy based thereon, wherein: a) at a PTH antagonist level above the normal range, therapy to increase the bone turnover rate in the person is started or increased, or therapy to decrease the bone turnover rate in the person is halted or decreased; b) at a PTH antagonist level below the normal range, therapy to decrease the bone turnover rate in the person is started or increased, or therapy to increase the bone turnover rate in the person is halted or decreased; and c) at a PTH antagonist level within the normal range, no bone turnover-related therapy is begun or altered.

In a related aspect, the therapy to decrease the bone turnover rate in the person comprises Vitamin D, Vitamin D analog, calcimimetic, calcium supplement therapy, PTH antagonist administration or a combination thereof. In a further related aspect, the therapy to increase the bone turnover rate in the person comprises administering PTH agonist, phosphate, calcililetic, EDTA, calcium binding agents, PTH, agents that stimulate PTH production or a combination thereof.

In one aspect the PTH agonist and PTH antagonist levels and the corresponding ratio are calculated using a Scantibodies Laboratory Whole PTH Assay, Scantibodies Laboratory CAP Assay, Scantibodies Laboratory Intact PTH Assay, Scantibodies Laboratory Total Intact PTH Assay or a combination thereof. PTH-related assays of this type are available from Scantibodies Laboratories, Santee Calif. In a related aspect, the non-Scantibodies Laboratory 3rd generation PTH assay or the non-Scantibodies Laboratory intact PTH assay is selected from the group consisting of Nichols Institute Diagnostics Allegro Intact PTH Assay, Nichols Institute Diagnostics Advantage Bio-Intact PTH Assay, Nichols Institute Advantage Intact PTH Assay, Immutopics, Inc. Human BioActive Intact PTH assay, Immutopics, Inc. Human Intact PTH assay, and the like. PTH-related assays of this second/third type are available from Nichols Institute Diagnostics, San Clemente, Calif.; and from Immutopics, Inc., San Clemente, Calif.

In a further embodiment, a method is provided for controlling the phosphate level a person comprising: obtaining a sample from a person to be tested; determining the level of a parathyroid hormone (PTH) agonist and a PTH antagonist; obtaining a ratio of the PTH agonist versus the PTH antagonist for the person; and controlling the phosphate level in the patient based on an inverse correlation between the PTH agonist/antagonist ratio and the blood phosphate level, wherein when the PTH agonist/antagonist ratio increases, the blood phosphate level in the person decreases. In one aspect, the PTH antagonist level may be increased, wherein the phosphate level in the patient increases. In another aspect, the PTH antagonist level may be decreased, wherein the phosphate level in the patient decreases. Frequently, the phosphate level may be adjusted through a method comprised of determining the ratio between PTH agonist and PTH antagonist in the person, and adjusting the PTH antagonist level to keep the product of the calcium and phosphate levels in the patient below about 55 mg²/ml².

In another aspect of the present invention, the patient population comprise a dialysis population comprised of end-stage renal disease (ESRD) and pre-ESRD patients.

### BRIEF DESCRIPTION OF THE DRAWINGS

FIG. 1 is a diagrammatic view of hPTH.

FIG. 2 illustrates comparison of the recognition of hPTH 1-84 and hPTH 7-84 by the Nichols Allegro Intact PTH assay. The Nichols 1-PTH assay does not differentiate between hPTH 1-84 (solid line) and hPTH 7-84 (dashed line).

FIG. 3 illustrates comparison of the recognition of hPTH 1-84 and hPTH 7-84 by the Scantibodies Whole PTH assay or the Scantibodies CAP PTH assay. Unlike the Nichols I-PTH assay, the Whole PTH assay does discriminate between hPTH 1-84 (solid line) and hPTH 7-84 (dashed line). Concentrations of hPTH 7-84 as high as 10,000 pg were undetectable in the Scantibodies CAP assay.

FIG. 4 presents a graph indicating a correlation between Nichols Bio-Intact Assay values and Scantibodies CAP Assay values for PTH₁₋₈₄. Based on the R² value, there is a significant correlation between these assays. However, the slope of the line indicates that there is not a significant assay bias between these two particular assays.

FIG. 5 presents a graph indicating a correlation between particular Nichols Intact PTH Assay values and Scantibodies Intact PTH Assay values for total PTH levels. Based on the R² value, there is a significant correlation between these assays. In addition, there is a significant assay bias between the Nichols intact PTH assay and the Scantibodies total intact PTH assay as indicated by the slope of the line.

FIG. 6 presents a graph indicating that no specific correlation exists between ratios generated between PTH agonist and PTH antagonist, by the particular Nichols assays and Scantibodies assays used in this example.

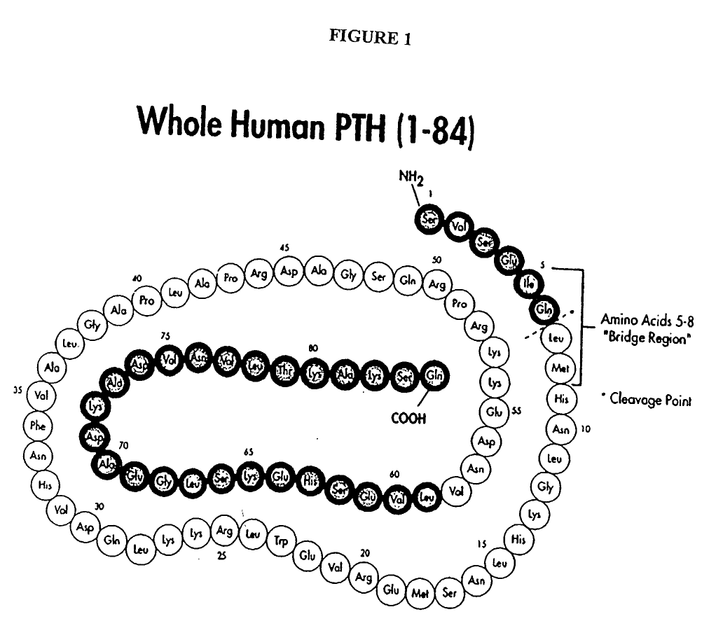

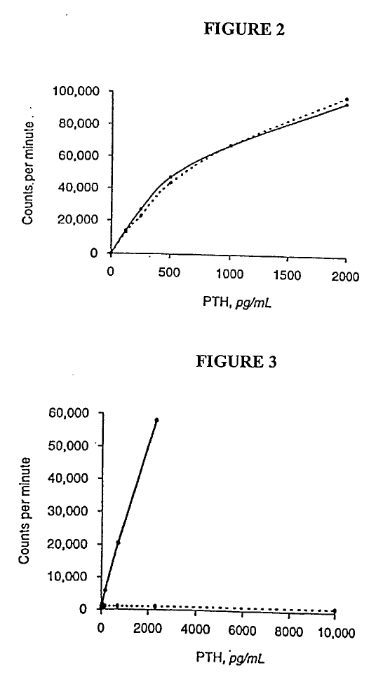

### DETAILED DESCRIPTION OF THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “parathyroid hormone (PTH) agonist” or “CAP” refers to the complete molecule of PTH or a fragment, derivative or analog thereof that stimulates osteoclasts formation and bone turnover to increase blood calcium levels. PTH agonist further refers to peptides which have PTH agonist properties. Other names of PTH include parathormone and parathyrin. For purposes herein, the name “parathyroid hormone (PTH)” is used herein, although all other names are contemplated. It is intended to encompass PTH agonist with conservative amino acid substitutions that do not substantially alter its biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., MOLECULAR BIOLOGY OF THE GENE, 4th Edition, 1987, The Bejamin/Cummings Pub. co., p.224). PTH agonist assay values may be obtained by measuring a sample with a Scantibodies Whole PTH Assay or a Scantibodies CAP Assay or a 3rd generation PTH Assay or a Nichols BioIntact PTH assay or an Immutopics Human Bioactive PTH assay.

As used herein, “parathyroid hormone (PTH) antagonist” or “CIP” refers to a PTH fragment or derivative that counters the effect of a PTH agonist or otherwise lacks PTH agonist activity. It is intended to encompass PTH antagonist with conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al. MOLECULAR BIOLOGY OF THE GENE, 4th Edition, 1987, The Bejacmin/Cummings Pub. co., p.224).

As used herein, the terms “total PTH,” “intact PTH” and “total intact PTH” are interchangeable and refer to an assay directed at measuring PTH agonist and PTH antagonist levels.

As used herein, a “functional derivative or fragment” of PTH agonist or PTH antagonist refers to a derivative or fragment of PTH that still substantially retains its function as a PTH agonist or PTH antagonist. Normally, the derivative or fragment retains at least 50% of its PTH agonist or PTH antagonist activity. Preferably, the derivative or fragment retains at least 60%, 70%, 80%, 90%, 95%, 99% and 100% of its PTH agonist or PTH antagonist activity. It is also possible that a functional derivative or fragment of PTH agonist or PTH antagonist has higher PTH agonist or PTH antagonist activity than a parent molecule from which the functional derivative or fragment is derived from.

As used herein, “Comparing the ratio of PTH agonist versus PTH antagonist to a list of probabilities” refers to: 1) a comparative value between PTH agonist and PTH antagonist in a individual mammal, e.g., human, that is statistically higher or lower than such a comparative value in the same individual mammal in a healthy state; 2) a comparative value between PTH agonist and PTH antagonist in a individual mammal, e.g., human, that is statistically higher or lower than such a comparative value in another comparable individual mammal in a healthy state; or 3) a comparative value between PTH agonist and PTH antagonist in a individual mammal, e.g., human, that is statistically higher or lower than a mean or average comparative value of comparable healthy population. As further used herein, the ratio may be adjusted prior to comparison with a list of probabilities. The difference between the PTH agonist/PTH antagonist ratio and the list of probabilities must be statistically significant so that the difference of the ratio and probabilities can be used in prognosis, diagnosis or treatment monitoring. The comparative value between PTH agonist and PTH antagonist can take any suitable form. For example, the comparative value can be a ratio, e.g., PTH agonist/PTH antagonist, PTH antagonist/PTH agonist, PTH agonist/the sum of PTH agonist and PTH antagonist, or PTH antagonist/the sum of PTH agonist and PTH antagonist, etc. In another example, the comparative value can be a subtraction value, e.g., PTH agonist-PTH antagonist, PTH antagonist-PTH agonist, etc. The above examples are for illustration only and are not intended to be an exhaustive list of all possible formats for measuring the comparative value between PTH agonist and PTH antagonist. Other suitable formats are readily apparent to skilled artisans and can be used.

In one example, the ratio between PTH agonist and PTH antagonist is determined by determining and comparing at least two of the parameters selected from the group consisting of the level of the PTH agonist, the PTH antagonist and the total PTH level, i.e., a sum of PTH agonist and PTH antagonist. In another example, the subject to be treated has a PTH agonist/PTH antagonist ratio less than or equal to 2.5. In still another example, the subject, e.g., a human, has PTH agonist-PTH antagonist value that equals or is less than 50 pg/ml. In yet another example, the subject, e.g., a human, has a PTH antagonist level that is more than the PTH agonist level.

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, “adynamic low bone turnover disease” refers to a variety of disorders involving abnormal PTH agonist and/or antagonist levels in a person. This definition is non-limiting in that it does not refer to only one specific disease, it refers to a variety of disorders that may result from abnormal PTH or PTH component levels in a person. As PTH levels are tied to bone turnover rate, abnormally low levels of PTH agonist, abnormally low levels of PTH agonist/antagonist ratios, and abnormally high levels of PTH antagonist may lead to abnormally low bone turnover in a person. In a person, this type of state may indicate the presence of, or susceptibility to, an adynamic low bone turnover disease. Conversely, abnormally high levels of PTH agonist, abnormally high levels of PTH agonist/antagonist ratios, and abnormally low levels of PTH antagonist may lead to abnormally high bone turnover in a person.

As used herein, “comparing a PTH agonist level by two PTH assays” and “comparing a total PTH level by two PTH assays” generally refers to equating analogous PTH assay values with one another. Total PTH values, PTH agonist values and/or PTH antagonist values may be compared by the methods described herein. In addition, ratios between total PTH values, PTH agonist values and PTH antagonist values may be compared by the disclosed methods. Without being bound by theory, a comparison may often come in the form of dividing, multiplying, adding and/or subtracting one value by the other analogous value. Occasionally, a comparison, as used herein, may refer to an overall comparison between analogous assays involving one or more multi-determinative components.

As used herein, “adjusting the total PTH level,” “adjusting the PTH agonist level,” and “adjusting the PTH antagonist level” generally refers to artificially modifying, transforming or converting the value or level obtained for a particular PTH assay to generate a value or level that is comparable to values from a reference assay to examine if the assay values in question fall within a range generated by an analogous PTH assay. A bias factor is generally used in this conversion. Total PTH values, PTH agonist values and/or PTH antagonist values may be adjusted by the methods described herein. In addition, ratios between total PTH values, PTH agonist values and PTH antagonist values may be adjusted by the disclosed methods. Generally, PTH-related value adjustments are performed with respect to a particular assay, which assay may comprise a proprietary assay such as PTH assays described herein produced by Scantibodies Laboratories.

As used herein, “adjusted ratio” refers to a ratio between PTH agonist and PTH antagonist comprised of an adjusted PTH agonist level and an adjusted PTH antagonist level. An adjusted ratio may also refer to a ratio between PTH antagonist and PTH agonist comprised of an adjusted PTH antagonist level and an adjusted PTH agonist level. An adjusted ratio may also refer to a ratio between total PTH and PTH agonist comprised of an adjusted total PTH level and an adjusted PTH agonist level. A variety of other ratio combinations are also contemplated between adjusted total PTH values, adjusted PTH agonist values and adjusted PTH antagonist values. Other suitable formats are readily apparent to skilled artisans and can be used.

As used herein, “bias factor” refers to a differential valuation between similar tests for a particular PTH assay component as determined by two or more assays. For example, a bias factor may comprise a factor representing the difference between a total PTH value for a sample as determined by one assay and the total PTH value determined by a second assay for the same sample. A bias factor may also refer to a factor representing the difference in values obtained between two different assays that are designated as specific for one or more particular PTH components. Generally, a bias factor is useful for converting or adjusting the value obtained by one of two assays to a value equivalent with the other assay. For example, a bias factor may be useful for obtaining an adjusted assay value for a first assay that approximates the value obtained for the same value by an analogous second assay through multiplying the bias factor, calculated between the first and second assay, by the first assay value. Bias factors may be obtained with respect to one assay and used for future conversions or adjustments of the assay values from that assay. For example, a bias factor may be obtained between two particular PTH assays after one or a series of comparisons; the bias factor obtained through these comparisons may be useful to convert or modify future PTH assay valuations. Without limitation, bias factors may be generated between values obtained for total PTH values, PTH agonist values, and PTH antagonist values. Bias values between PTH antagonist levels may be generated, for example, for between assays capable of directly detecting PTH antagonist; in this circumstance a bias factor between PTH agonist/antagonist ratios may also be generated. The above examples are for illustration only and are not intended to be an exhaustive list of all possible formats generating a bias factor. Other suitable formats are readily apparent to skilled artisans and can be used.

Bias factors of the present invention may be obtained by comparing valuations obtained by two or more assays that represent similar tests for a particular PTH assay component. Frequently, a bias factor may be determined by dividing one of the values obtained for a particular PTH assay component using a series of samples through the practice of a first assay by a value obtained for the same PTH assay component using the same series of samples through the practice of a second assay. For example, a total PTH level for a person may be obtained by a first assay for a particular series of samples; subsequently or concurrently, a total PTH level for the same series of samples may be obtained by a second assay. Further, to determine a bias factor between these two total PTH values one would divide each of the assay values obtained from one assay by the corresponding assay value obtained in the second assay to obtain a bias factor; the mean of all of the bias factors would be the assay bias factor. For example, in the scenario presented above, the total PTH value obtained by the first assay may be used to divide the total PTH value obtained by the second assay to obtain a bias factor that would be useful to convert the total PTH value obtained by the second assay to the total PTH value obtained by the first assay.

As used herein, the term “target range” refers to a PTH agonist/antagonist, PTH agonist, or PTH antagonist range present in a particular patient population. The target range includes PTH agonist/antagonist, PTH agonist, or PTH antagonist ranges for a variety of persons without regard to limiting factors such as gender, race, age, etc. Therefore, as bone turnover rates vary between selected individuals depending on such limiting factors, the normal PTH range for persons in one group (e.g., an adult) may not be the same as the normal PTH range for other persons in a different group (e.g., a child). Therefore, the target range incorporates a variety of “normal ranges” within its bounds. It should be noted that a variety of combinations of groups are possible and the normal PTH ranges for these groups may vary accordingly. Example 2 further elaborates on the basis of target ranges.

As used herein, the term “normal range” refers to a normal PTH agonist/antagonist, PTH agonist, or PTH antagonist range present in a particular dialysis patient population. Generally, a normal range lies within the bounds of the target range discussed above although it does not comprise the total target range. While not bound by theory, normal ratio ranges may frequently span about one unit in length. For example, a typical normal range for a PTH agonist/antagonist ratio, including adjusted ratios, may lie between 1.17 and 2.17, 1.5 and 2.5, 2 and 3, 2.15 and 3.15, although a variety of range modifications may be included depending on the person. Examples 2-4 further elaborate on the basis of normal ranges.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

B. Parathyroid Hormone Antagonists

In one aspect, the present invention is directed to a parathyroid hormone (PTH) antagonist, which PTH antagonist comprises a contiguous portion of human PTH having an amino acid sequence set forth in SEQ ID NO:1 (PTH₁₋₈₄), or a nucleic acid encoding said portion of human PTH, and said PTH antagonist has the following characteristics: a) the N-terminal amino acid residue of said PTH antagonist starts at any position spanning position 2 through position 33 of said PTH₁₋₈₄; b) the C-terminal amino acid residue of said PTH antagonist ends at any position spanning position 35 through position 84 of said PTH₁₋₈₄; and c) said PTH antagonist has a minimal length of three amino acid residues. Preferably, the PTH antagonist is in the form of a pharmaceutical composition, which pharmaceutical composition comprises an effective amount of the PTH antagonist and a pharmaceutically acceptable carrier or excipient.

The N-terminal amino acid residue of the PTH antagonist can start at any position spanning position 2 through position 33 of said PTH₁₋₈₄. For example, the N-terminal amino acid residue of the PTH antagonist can start at position 2 of the PTH₁₋₈₄. The C-terminal amino acid residue of said PTH antagonist can end at any position spanning position 35 through position 84 of said PTH₁₋₈₄. For example, the C-terminal amino acid residue of the PTH antagonist can end at position 84 of the PTH₁₋₈₄.

In a specific embodiment, the PTH antagonist is a protein or a peptide, or a nucleic acid encoding said protein or peptide, selected from the group consisting of PTH₂₋₈₄, PTH₃₋₈₄, PTH₄₋₈₄, PTH₅₋₈₄, PTH₆₋₈₄, PTH₇₋₈₄, PTH₈₋₈₄, PTH₉₋₈₄, PTH₁₀₋₈₄, PTH₁₁₋₈₄, PTH₁₂₋₈₄, PTH₁₃₋₈₄, PTH₁₄₋₈₄, PTH₁₅₋₈₄, PTH₁₆₋₈₄, PTH₁₇₋₈₄, PTH₁₈₋₈₄, PTH₁₉₋₈₄, PTH₂₀₋₈₄, PTH₂₁₋₈₄, PTH₂₂₋₈₄, PTH₂₃₋₈₄, PTH₂₄₋₈₄, PTH₂₅₋₈₄, PTH₂₆₋₈₄, PTH₂₇₋₈₄, PTH₂₈₋₈₄, PTH₂₉₋₈₄, PTH₃₀₋₈₄, PTH₃₁₋₈₄, PTH₃₂₋₈₄, and PTH₃₃₋₈₄. In another specific embodiment, the PTH antagonist is a protein or a peptide, or a nucleic acid encoding said protein or peptide, selected from the group consisting of PTH₇₋₆₉, PTH₇₋₇₀, PTH₇₋₇₁, PTH₇₋₇₂, PTH₇₋₇₃, PTH₇₋₇₄, PTH₇₋₇₅, PTH₇₋₇₆, PTH₇₋₇₇, PTH₇₋₇₈, PTH₇₋₇₉, PTH₇₋₈₀, PTH₇₋₈₁, PTH₇₋₈₂, PTH₇₋₈₃ and PTH₇₋₈₄.

The PTH antagonist can have any suitable length provided that it maintains its antagonizing activity. For example, the PTH antagonist can have a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 or 83 amino acid residues.

The PTH antagonist can further comprise an amino acid residue substitution or modification that enhances or does not decrease its antagonist activity, or an amino acid residue substitution or modification that stabilizes the PTH antagonist. For example, the PTH antagonist can further comprise the following amino acid residue substitution or modification: His₂₅, His₂₆, Leu₂₇, (U.S. Pat. No. 5,382,658); Tyr₃₄, D-Trp₁₂, Nle_{8,18}, desamino(Nle_{8,18}), Lys₁₃ modified in the epsilon-amino acid group by N,N-diisobutyl or 3-phenylpropanoyl (U.S. Pat. No. 5,093,233); Gly₁₂ substituted by D-Trp, L-Trp, L- or D-α- or β-naphthylalanine, or D- or L-α-MeTrp (U.S. Pat. No. 4,968,669); the amino acid residue at positions 7, 11, 23, 24, 27, 28, or 31 being cyclohexylalanine, the amino acid residue at position 3, 16, 17, 18, 19, or 34 being α-aminoisobutyric acid, the amino acid residue at position 1 being α,β-diaminopropionic acid, the amino acid residue at position 27 being homoarginine, the amino acid residue at position 31 being norleucine (U.S. Pat. No. 5,723,577); each of Arg₂₅, Lys₂₆, Lys₂₇ being substituted with Ala, Asn, Asp, Cys, Gin, Glu, Gly, His, Ile, Leu, Met, Phe, Pro, Ser, Thr, Trp, Tyr or Val (U.S. Pat. No. 5,317,010); and a combination thereof.

C. Parathyroid Hormone Agonists

In one aspect, the present invention is directed to a parathyroid hormone (PTH) agonist, which PTH agonist comprises a contiguous portion of human PTH having an amino acid sequence set forth in SEQ ID NO: 1 (PTH₁₋₈₄), and the PTH agonist has the following characteristics: a) the N-terminal amino acid residue of the PTH agonist starts at position 1 of the PTH₁₋₈₄; and b) the C-terminal amino acid residue of the PTH agonist ends at any position spanning position 34 through position 84 of the PTH₁₋₈₄.

Without being bound by theory, the N-terminal amino acid residue of the PTH agonist generally starts at position 1 of said PTH₁₋₈₄. For example, the N-terminal amino acid residue of the PTH agonist can start at position 1 of the PTH₁₋₈₄. The C-terminal amino acid residue of said PTH agonist can end at any position spanning position 34 through position 84 of said PTH₁₋₈₄. For example, the C-terminal amino acid residue of the PTH agonist can end at position 84 of the PTH₁₋₈₄.

The PTH agonist can have any suitable length provided that it maintains its agonizing activity. For example, the PTH agonist can have a length of 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 or 83 amino acid residues.

PTH agonists may comprise whole PTH, see, for example the peptides in U.S. Pat. Nos. 5,496,801, 5,208,041 or 4,086,196. Suitable PTH agonists may be derived from a variety of mammal species, for example bovine₁₋₃₅ and porcine₁₋₃₆ PTH peptide fragments (U.S. Pat. No. 5,783,558). The PTH agonist can further comprise an amino acid residue substitution or modification that enhances or does not decrease its agonist activity, or an amino acid residue substitution or modification that stabilizes the PTH agonist (see e.g., U.S. Pat. No. 5,382,658 (including His₂₅, His₂₆, and Leu₂₇ modifications)). PTH agonists, therefore, may comprise peptides which are structural analogs or fragments of a naturally occurring PTH (see e.g., U.S. Pat. No. 5,434,246 (including substitutions at the PTH 3, 14, 15, 16, 17, 25, 26, 27 or 34 amino acid positions); U.S. Pat. No. 4,656,250 (including PTH analogs with substitutions at the 8, 18 and 34 positions)). Synthetic polypeptide analogs of PTH, parathyroid hormone related peptide (PTHrp), and of the physiologically active truncated homologs and analogs of PTH and PTHrp, in which amino acid residues (22-31) form an amphipathic α-helix, said residues (22-31) selected from hydrophilic amino acids (Haa) and lipophilic amino acids (Laa) ordered in the sequence: Haa(Laa Laa Haa Haa)₂ Laa and their pharmaceutically acceptable salts. See U.S. Pat. Nos. 5,807,823; 5,840,831; 5,798,225; 5,695,955; and 5,589,452. Moreover, PTH agonists may also include synthetic peptides, i.e., parathyroid hormone-like protein (PLP), or naturally occurring peptides such as (PTH)-like hypercalcemic factor (hHCF), parathyroid-related protein (PTHrP), or parathyroid hormone-like adenylate cyclase-stimulating proteins (hACSPs). See, e.g, Yates, A J, et al., J. Clin. Invest. (1988) 81(3):932-8; Nissenson R A, et al., J. Biol. Chem. (1988) 263(26):12866-71; Thompson D D, et al., Proc. Nat'l Acad. Sci. (1988) 85(15):5673-7; and Stewart, A F, et al., J. Clin. Invest. (1988) 81(2):596-600. For example, the PTH agonist can further comprise the following amino acid residue substitution or modification of PTH, PLP, or PTHrP: each of Ser₃, Gln₆, His₉ being substituted with Ala, Arg, Asn, Asp, Cys, Gln, Glu, Gly, His, Ile, Leu, Lys, Met, Phe, Pro, Ser, Thr, Trp, Tyr or Val (U.S. Pat. No. 5,849,695). Other PTH and PTHrP agonists contemplated by the present disclosure may also include human PTH (hPTH)₁₋₃₄NH₂, hPTH₁₋₃₈NH₂, hPTH₁₋₄₄NH₂, hPTH₁₋₆₈NH₂, [Nle^{8,18},Tyr³⁴]bPTH₁₋₃₄NH₂, bPTH₁₋₃₄NH₂, [Nle^{8,18},Tyr³⁴]bPTH₁₋₃₄, [Nle^{8,18},Phe²²,Tyr³⁴]bPTH₁₋₃₄NH₂, [Nle^{8,18},Arg¹⁹,Tyr³⁴]bPTH₁₋₃₄NH₂, [Nle^{8,18},Arg²¹,Tyr³⁴]bPTH₁₋₃₄NH₂, or [Nle^{8,18},Arg_{19,21},Tyr³⁴]bPTH₁₋₃₄NH₂. The symbol NH₂ denotes amidation of the carboxyl group (—CO.OH) of the C-terminal amino acid to form —CO.NH₂. See U.S. Pat. No. 5,747,456.

D. Methods for Assessing a Person's Bone Turnover Rate

The present invention is also directed to a method for identifying a subject having or at risk of having an adynamic low bone turnover disease, which method comprises determining PTH antagonist level or a comparative value between PTH agonist and PTH antagonist and identifying a subject having an abnormal PTH antagonist level or an abnormal comparative value between PTH agonist and PTH antagonist as having or at risk of having an adynamic low bone turnover disease. Frequently, therapy or treatment decisions may be based on the levels determined for total PTH or intact PTH. The present disclosure presents the use of total PTH, PTH agonist, PTH antagonist, comparisons and combinations thereof, as well as adjusted levels of these components for therapy and/or treatment decisions.

PTH Agonist/Antagonist Ratio

In one aspect, a method is provided for assessing a person's bone turnover rate comprising: a) obtaining a sample from a person to be tested; b) determining the level of a parathyroid hormone (PTH) agonist and a PTH antagonist in the sample; c) obtaining a ratio of the PTH agonist versus the PTH antagonist for the person; and d) comparing the ratio obtained in step c) to a list of probabilities for predicting adynamic low bone turnover disease expressed as a percentage for accurate prediction by a reference method (i.e., bone histology) of an adynamic low bone turnover disease, the probabilities being in a relationship to PTH agonist/antagonist ratios based on a dialysis patient population, wherein the population has a clinically significant risk of an adynamic low bone turnover disease at particular ratio ranges. In a related aspect, the PTH antagonist level is determined by determining a total PTH level and determining a PTH agonist level followed by subtracting the PTH agonist level from the total PTH level.

Frequently, the PTH agonist and PTH antagonist levels and the corresponding ratio may be calculated using a Scantibodies Laboratory Whole PTH Assay, Scantibodies Laboratory CAP Assay, Scantibodies Laboratory Intact PTH Assay, Scantibodies Laboratory Total Intact PTH Assay or a combination thereof. PTH-related assays of this type are available from Scantibodies Laboratories, Santee Calif.

In a particular aspect, a person may have a clinically significant risk of an adynamic low bone turnover disease at PTH agonist/PTH antagonist ratios of less than about 1.17 to about 3.15. Frequently, a person having a clinically significant risk of adynamic low bone turnover disease may have a PTH agonist/PTH antagonist ratio below about 1.17, below about 1.80, below about 2.5, and below about 3.15. The clinical significance of having an adynamic low bone turnover disease may also be present, for example, for a person having a PTH agonist/PTH antagonist ratio within or below ranges 1.17 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.15. Depending on the particular person (i.e., age, sex, geographical location, ethnicity, etc.) the normal PTH agonist/antagonist range may vary in terms of overall values and spread. Accordingly, the values provided above are for illustrative purposes only.

In another aspect, a person may be determined as having a normal or high bone turnover rate at a ratio of above about 3.15. On occasion, depending on the particular person, the person may be classified as having a normal or high bone turnover rate at ratios of above 1.17, above about 1.80 and above 3.15. For example, a person may have a high bone turnover rate at ratios ranges above about 1.17 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.15.

In one aspect, the present description contemplates a variety of PTH assays. Frequently, PTH assays of the present invention comprise immunoassays. A variety of immunoassays are contemplated for use in the presently described methods. Generally, however, the object of any given assay is to analyze the binding between an analyte, if present in a sample, and one or more immunoreactants. This analysis may be in sandwich assay or competitive assay format or antibody detection assay format. Representative assays may include, for example, an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, μ-capture assay, inhibition assay, energy transfer assay, avidity assay, turbidometric immunoassay and time resolved amplified cryptate emission (TRACE) assay.

A variety of patient populations may benefit from the present invention. Generally, without limitation, such populations may be dialysis patients, pre-dialysis patients, end-stage renal disease (ESRD) patients, pre end-stage renal disease (ESRD) persons, or osteoporosis patients. In a particular aspect of the present invention the patient population, for testing and reference purposes, comprises ESRD and/or pre-ESRD patients. As used herein, a patient refers to a person afflicted with, diagnosed, or otherwise suspected as having a particular disorder.

In another aspect, the patient population used to generate a predictive function of the risk of a person of having an adynamic low bone turnover disease is an end-stage renal disease (ESRD) patient population that has received some form of bone affecting treatments. In this population about 52% have a clinically significant risk of an adynamic low bone turnover disease at a ratio of less than about 1.17 to about 3.15. Frequently, the majority of the ESRD patient population has been subjected to Vitamin D, Vitamin D analog, calcimimetic, calcium supplement treatment or other related PTH suppression therapy treatment.

In yet another aspect, the parathyroid hormone agonist level of the present invention is determined using an antibody that distinguishes PTH agonist from PTH antagonist. In a related aspect, the parathyroid hormone antagonist level may be determined using an antibody that distinguishes PTH antagonist from PTH agonist. Suitable antibodies include those that are an antibody or an antibody fragment specific for the PTH peptide SER-VAL-SER-GLU-ILE-GLN (SEQ ID NO:2); or antibodies comprising an anti-(1-6) PTH antibody, anti-(1-4) PTH antibody, anti-(1-9) PTH antibody, anti-(1-11) PTH antibody, anti-(1-12) PTH antibody, or a combination thereof.

In another aspect, a method of guiding therapy for persons suspected of having an adynamic bone turnover disease is provided, comprising determining the PTH agonist/antagonist ratio and determining therapy based thereon, wherein bone turnover-related therapy is started, stopped or held constant if the ratio is less between about 1.17 to about 3.15. An example method of guiding therapy for persons suspected of having an adynamic low bone turnover disease comprises determining the PTH agonist/antagonist ratio and determining therapy based thereon, wherein: a) at a ratio below the normal ratio range, therapy to increase the bone turnover rate in the person is started or increased, or therapy to decrease the bone turnover rate in the person is halted or decreased; b) at a ratio above the normal ratio range, therapy to decrease the bone turnover rate in the person is started or increased, or therapy to increase the bone turnover rate in the person is halted or decreased; and c) at a ratio within the normal ratio range, no bone turnover-related therapy is begun or altered. In a related aspect, therapy to decrease the bone turnover rate in the person comprises Vitamin D or Vitamin D analog (e.g., Zemplar® or Rocatrol® available from Hoffman La-Roche, Inc.), calcimimetic, calcium supplement therapy, PTH antagonist administration or a combination thereof. In another related aspect, therapy to increase the bone turnover rate in the person comprises administering PTH agonist, phosphate, calcililetic, PTH, EDTA, calcium binding agents or stimulating PTH production or a combination thereof. See e.g., Goodman W G, Turner S A, Adv. Ren. Replace Ther. July 2002;9(3):200-8 (calcimimetic agents); Parthemore J G, et al., J. Clin. Endocrinol. Metab. August 1978;47(2):284-9 (EDTA).

Adjusted PTH Agonist/Antagonist Ratio

Also provided herein is a method for assessing a person's bone turnover rate through adjusting a PTH agonist/antagonist ratio (by performing an adjustment, through the use of a bias factor, on the total intact PTH and PTH agonist assay values before the calculation of the ratio) and comparing this ratio to a list of probabilities comprising: a) obtaining a sample from a person to be tested; b) determining and comparing a total PTH level by two assays to generate a total PTH bias factor, the assays comprising (1) a Scantibodies Laboratory Total Intact PTH Assay or a Scantibodies Laboratory Intact PTH Assay, or a combination thereof, and (2) a non-Scantibodies Laboratory intact PTH assay; c) determining and comparing a PTH agonist level by two PTH assays to generate a PTH agonist bias factor, the assays comprising (1) a Scantibodies Laboratory Whole PTH Assay or a Scantibodies Laboratory CAP Assay, or a combination thereof, and (2) a non-Scantibodies Laboratory 3^{rd} generation PTH assay; d) adjusting the total PTH level determined by the non-Scantibodies Laboratory intact PTH assay, whereby the total PTH bias factor is multiplied by the total PTH level determined by the non-Scantibodies Laboratory 3^{rd} generation PTH assay to obtain an adjusted total PTH level; e) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level; f) obtaining an adjusted PTH antagonist level by subtracting the adjusted PTH agonist level from the adjusted total PTH level; g) obtaining an adjusted ratio of the adjusted PTH agonist versus the adjusted PTH antagonist; and h) comparing the adjusted ratio to a list of probabilities expressed as a percentage for an adynamic low bone turnover disease, the probabilities being in a relationship to PTH agonist/antagonist ratios based on a dialysis patient population, wherein the population has a clinically significant risk of an adynamic low bone turnover disease below a normal PTH agonist/antagonist ratio range within a target ratio range of between about 1.17 to about 3.15, and wherein the person is determined as having an adynamic low bone turnover disease if the adjusted ratio of step g) is below the normal range within the target ratio range.

In a related aspect, frequently the total PTH bias factor is obtained by dividing the total PTH value obtained through the practice of the non-Scantibodies Laboratory PTH assay by the corresponding total PTH value obtained through the practice of the Scantibodies Laboratory Total Intact PTH Assay or a Scantibodies Laboratory Intact PTH Assay; and wherein the PTH agonist bias factor is obtained by dividing the PTH agonist value obtained through the practice of the non-Scantibodies Laboratory 3^{rd} generation PTH assay by the corresponding PTH agonist value obtained through the practice of the Scantibodies Laboratory Whole PTH Assay or a Scantibodies Laboratory CAP assay.

In one aspect, the non-Scantibodies Laboratory 3rd generation PTH assay or the non-Scantibodies Laboratory intact PTH assay may be an assay selected from the group consisting of Nichols Institute Diagnostics Allegro Intact PTH Assay and Nichols Institute Diagnostics Advantage Bio-Intact PTH Assay both available from Nichols Institute Diagnostics, San Clemente, Calif.; Immutopics Human BioActive Intact PTH assay and Immutopics Human Intact PTH assay, both available from Immutopics, Inc., San Clemente, Calif. In a related aspect, non-Scantibodies Laboratories 3rd generation PTH assays or non-Scantibodies Laboratory intact PTH assays may comprise variations and extensions of the above assays. The conversions, adjustments and ranges of present invention are compatible with a variety of PTH assays and are not limited to those described herein.

In a related aspect, the non-Scantibodies Laboratory 3rd generation PTH assay or the non-Scantibodies PTH assay is the same or different assay used to determine total PTH and PTH agonist levels.

In a particular aspect, a person may have a clinically significant risk of an adynamic low bone turnover disease at an adjusted PTH agonist/PTH antagonist ratio of less than about 1.17 to about 3.15. Frequently, a person may have an adjusted PTH agonist/PTH antagonist ratio below about 1.17, about 1.80, about 2.5, and about 3.15 and have a clinically significant risk of an adynamic low bone turnover disease. The clinical significance of having an adynamic low bone turnover disease may also be present, for example, for a person having an adjusted PTH agonist/PTH antagonist ratio within or below ranges 1.17 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.15. Depending on the particular person the normal adjusted PTH agonist/antagonist range may vary in terms of overall values and spread. Accordingly, the values provided above are for illustrative purposes only.

In another aspect, a person may be determined as having a normal or high bone turnover rate at an adjusted PTH agonist/antagonist ratio of above about 3.15. On occasion, depending on the particular person, the person may be classified as having a normal or high bone turnover rate at adjusted ratios of above 1.17, above about 1.80 and above 3.15. For example, a person may have a high bone turnover rate at adjusted ratios ranges above about 1.17 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.15.

In yet another aspect, a course or regimen of treatment may be dictated upon the adjusted PTH agonist/antagonist ratio. Bone turnover-related therapy, e.g., suppression therapy such as Vitamin D, Vitamin D analog, calcimimetic or calcium supplement therapy may be started, increased, held constant or stopped depending on the adjusted PTH agonist/antagonist ratio determined. In a related aspect, therapy to increase PTH production (e.g., exogenous PTH or phosphate administration or EDTA administration or calcium binder administration or PTH stimulating agent administration) may be started, increased, held constant or stopped depending on the adjusted PTH agonist/antagonist ratio determined.

In another aspect, a method of guiding therapy for persons suspected of having an adynamic bone turnover disease is provided, comprising determining the adjusted PTH agonist/antagonist ratio and determining therapy based thereon, wherein bone turnover-related therapy is started, stopped or held constant if the ratio is less between about 1.17 to about 3.15. An example method of guiding therapy for persons suspected of having an adynamic low bone turnover disease comprises determining the adjusted PTH agonist/antagonist ratio and determining therapy based thereon, wherein: a) at an adjusted ratio below the normal ratio range, therapy to increase the bone turnover rate in the person is started or increased, or therapy to decrease the bone turnover rate in the person is halted or decreased; b) at an adjusted ratio above the normal ratio range, therapy to decrease the bone turnover rate in the person is started or increased, or therapy to increase the bone turnover rate in the person is halted or decreased; and c) at an adjusted ratio within the normal ratio range, no bone turnover-related therapy is begun or altered. In a related aspect, therapy to decrease the bone turnover rate in the person comprises Vitamin D, Vitamin D analog, calcimimetic, calcium supplement therapy, PTH antagonist administration or a combination thereof. In another related aspect, therapy to increase the bone turnover rate in the person comprises administering PTH agonist, phosphate, EDTA, calcium binding agents, calcililetic, or stimulating PTH production, or a combination thereof.

Risk and Therapy Determinations Based on PTH Agonist Levels and PTH Antagonist Levels

In one aspect of the present invention, when the rate of bone turnover is not optimal because it is too high (i.e., high bone turnover disease) it is due to a PTH agonist level that is too high relative to the level of PTH antagonist or the PTH agonist/PTH antagonist ratio is too high or the PTH antagonist level is too low relative to the level of PTH agonist. When the rate of bone turnover is not optimal because it is too low (i.e., adynamic low bone turnover disease) it is because the level of PTH antagonist is too high relative to the level of PTH agonist or the PTH agonist/PTH antagonist ratio is too low or PTH agonist is too low relative to the level of PTH antagonist. Unique patient groups have reference ranges (“normal ranges” for the particular group) of PTH agonist, PTH antagonist and PTH agonist/PTH antagonist ratio uniquely associated with them that correspond to ranges for adynamic low bone turnover, normal bone turnover and high bone turnover states. These unique patient groups, and their corresponding reference ranges, are often differentiated from one another based on disease states and stages (i.e., ESRD vs. osteoporosis), gender, age groups, geographical location (i.e., sunny localities contributing to higher vitamin D levels), ethnicity, diet, nutritional status, vitamin D levels, etc. The minor variations of reference ranges in these groups are represented by the normal ranges contained within the target ranges of the present invention. Therefore, the present invention claims the measurement of PTH agonist (as opposed to the total “intact” PTH that has previously been used), PTH antagonist and the PTH agonist/PTH antagonist ratio in order to diagnose the bone turnover status for patients afflicted by these bone diseases (e.g., renal failure patients, post menopausal women, osteoporosis patients, newborns, nutritionally challenged persons).

In another aspect, after the initial determination of bone turnover rate has been made, the present invention further provides the use of PTH agonist, PTH antagonist, and PTH agonist/PTH antagonist measurements to monitor or guide when interventional therapy is given to these patients. Interventional therapy that changes the levels of PTH agonist, PTH antagonist and PTH agonist/PTH antagonist ratio in these patients falls into two categories comprising direct and indirect change of the PTH agonist, PTH antagonist and PTH agonist/PTH antagonist ratio. Direct intervention may comprise the administration of PTH agonist and PTH antagonist to the patient. Indirect intervention may comprise the administration of agents that will change the parathyroid gland's secretion of PTH agonist and PTH antagonist. Indirect intervention agents are further described herein but may comprise Vitamin D, Vitamin D analogues, calcium, phosphate, calcimimetics and calcililetics.

The present disclosure also provides methods for determining PTH agonist and/or PTH antagonist levels for a person and determining the risk of that person for having an adynamic low bone turnover disease. In a related aspect, methods are provided herein for determining PTH agonist and/or PTH antagonist levels for a person and determining or modifying treatment courses or regimen based on such determinations.

In one aspect, a method for assessing a person's bone turnover rate is provided, comprising: a) obtaining a sample from a person to be tested; b) determining the level of a parathyroid hormone (PTH) agonist; and c) comparing the PTH agonist level to a list of probabilities for predicting adynamic low bone turnover disease expressed as a percentage for accurate prediction of an adynamic low bone turnover disease, the probabilities being in a relationship to PTH agonist levels based on a dialysis patient population, wherein the population has a clinically significant risk of an adynamic low bone turnover disease below a normal PTH agonist range within a target PTH agonist range of between about 83 pgm/ml to about 412 pgm/ml, and wherein the person is determined as having an adynamic low bone turnover disease if the PTH agonist level is below the normal range within the target range. Frequently, the person may have a clinically significant risk of an adynamic low bone turnover disease at a PTH agonist level of below about 127 pgm/ml.

In a related aspect, the PTH agonist level may be an adjusted PTH agonist level that was adjusted through the use of a corresponding PTH agonist bias factor. Such method may comprise: a) obtaining a sample from a person to be tested; b) determining and comparing a PTH agonist level by two PTH assays to generate a PTH agonist bias factor, the assays comprising (1) a Scantibodies Laboratory Whole PTH Assay or a Scantibodies Laboratory CAP Assay, or a combination thereof, and (2) a non-Scantibodies Laboratory 3^{rd} generation PTH assay; c) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3 generation PTH assay to obtain an adjusted PTH agonist level; and d) comparing the adjusted PTH agonist level to a list of probabilities for predicting adynamic low bone turnover disease expressed as a percentage for accurate prediction of an adynamic low bone turnover disease, the probabilities being in a relationship to PTH agonist levels based on a dialysis patient population, wherein the population has a clinically significant risk of an adynamic low bone turnover disease below a normal PTH agonist range within a target PTH agonist range of between about 83 pgm/ml to about 412 pgm/ml, and wherein the person is determined as having an adynamic low bone turnover disease if the adjusted PTH agonist level is below the normal range within the target range. Frequently, the person may have a clinically significant risk of an adynamic low bone turnover disease at an adjusted PTH agonist level of below about 127 pgm/ml.

In another aspect, methods of guiding therapy for persons suspected of having an adynamic bone turnover disease are provided, such methods may be comprised of determining the PTH agonist level and determining therapy based thereon, wherein bone turnover-related therapy is started, stopped or held constant if the PTH agonist level is between about 83 pgm/ml to about 412 pgm/ml. Frequently, the PTH suppression therapy may be started, stopped or held constant if the PTH agonist level is below a value between about 83 pgm/ml to about 127 pgm/ml. Such methods may comprise determining the PTH agonist level and determining therapy based thereon, wherein: a) at a PTH agonist level below the normal ratio range, therapy to increase the bone turnover rate in the person is started or increased, or therapy to decrease the bone turnover rate in the person is halted or decreased; b) at a PTH agonist level above the normal ratio range, therapy to decrease the bone turnover rate in the person is started or increased, or therapy to increase the bone turnover rate in the person is halted or decreased; and c) at a PTH agonist level within the normal ratio range, no bone turnover-related therapy is begun or altered. In a related aspect, the PTH agonist level may be an adjusted PTH agonist level that was adjusted through the use of a corresponding PTH agonist bias factor.

In another aspect, a method for assessing a person's bone turnover rate is provided, comprising: a) obtaining a sample from a person to be tested; b) determining the level of a parathyroid hormone (PTH) antagonist; and c) comparing the PTH antagonist level to a list of probabilities for predicting adynamic low bone turnover disease expressed as a percentage for accurate prediction of an adynamic low bone turnover disease, the probabilities being in a relationship to PTH antagonist levels based on a dialysis patient population, wherein the population has a clinically significant risk of an adynamic low bone turnover disease above a normal PTH antagonist range within a target PTH antagonist range of between about 14 pgm/ml to about 91 pgm/ml, and wherein the person is determined as having an adynamic low bone turnover disease if the PTH antagonist level is above the normal range within the target range. Frequently, the person may have a clinically significant risk of an adynamic low bone turnover disease at a PTH agonist level of above about 63 pgm/ml. In a related aspect, the PTH antagonist level may be an adjusted PTH antagonist level. Adjusted PTH antagonist levels may be determined through methods described hereinbefore, including through subtracting an adjusted PTH agonist level from a corresponding adjusted total PTH level, and/or separately through the use of a PTH antagonist bias factor as described below.

In another embodiment, methods are provided for assessing a person's bone turnover rate comprising: a) obtaining a sample from a person to be tested; b) determining and comparing a total PTH level by two assays to generate a total PTH bias factor, the assays comprising (1) a Scantibodies Laboratory Total Intact PTH Assay or a Scantibodies Laboratory Intact PTH Assay, or a combination thereof, and (2) a non-Scantibodies Laboratory intact PTH assay; c) determining and comparing a PTH agonist level by two PTH assays to generate a PTH agonist bias factor, the assays comprising (1) a Scantibodies Laboratory Whole PTH Assay or a Scantibodies Laboratory CAP Assay, or a combination thereof, and (2) a non-Scantibodies Laboratory 3^{rd} generation PTH assay; d) adjusting the total PTH level determined by the non-Scantibodies Laboratory intact PTH assay, whereby the total PTH bias factor is multiplied by the total PTH level determined by the non-Scantibodies Laboratory intact PTH assay to obtain an adjusted total PTH level; e) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level; f) obtaining an adjusted PTH antagonist level by subtracting the adjusted PTH agonist level from the adjusted total PTH level; and g) comparing the PTH antagonist level to a list of probabilities for predicting adynamic low bone turnover disease expressed as a percentage for accurate prediction (by a reference method such as bone histology) of an adynamic low bone turnover disease, the probabilities being in a relationship to PTH antagonist levels based on a dialysis patient population, wherein the population has a clinically significant risk of an adynamic low bone turnover disease above a normal PTH antagonist range within a target PTH antagonist range of between about 14 pgm/ml to about 91 pgm/ml, and wherein the person is determined as having an adynamic low bone turnover disease if the PTH antagonist level is above the normal range within the target range. Frequently, the person may have a clinically significant risk of an adynamic low bone turnover disease at an adjusted PTH antagonist level of above about 63 pgm/ml.

In another aspect, methods of guiding therapy a person suspected of having an adynamic bone turnover disease are provided, such methods may be comprised of determining the PTH agonist level and determining therapy based thereon, wherein bone turnover-related therapy is started, stopped or held constant if the PTH antagonist level is between about 14 pgm/ml to about 91 pgm/ml. Frequently, the PTH suppression therapy may be started, stopped or held constant if the PTH antagonist level is about 63 pgm/ml. Such methods may comprise determining the PTH antagonist level and determining therapy based thereon, wherein: a) at a PTH antagonist level above the normal ratio range, therapy to increase the bone turnover rate in the person is started or increased, or therapy to decrease the bone turnover rate in the person is halted or decreased; b) at a PTH antagonist level below the normal ratio range, therapy to decrease the bone turnover rate in the person is started or increased, or therapy to increase the bone turnover rate in the person is halted or decreased; and c) at a PTH antagonist level within the normal ratio range, no bone turnover-related therapy is begun or altered. In a related aspect, the PTH antagonist level may be an adjusted PTH antagonist level.

Target and Normal Ranges

The present invention contemplates the use of numerical ranges in determining risk of a bone turnover-related disorder and status with regard to a particular bone turnover-related disorder. The following Table 1 is provided to illustrate target ranges of the present invention and includes adjusted ratios and levels.

TABLE 1

| Target | Lower limit | Mid-point | Upper limit |
|---|---|---|---|
| PTH agonist/antagonist (ratio) | 1.17 | 1.8 | 3.15 |
| PTH agonist (level) | 83 pgm/ml | 127 pgm/ml | 412 pgm/ml |
| PTH antagonist (level) | 14 pgm/ml | 63 pgm/ml | 91 pgm/ml |

Target ranges of the present invention refer to range levels of PTH agonist/antagonist, PTH agonist, or PTH antagonist present in a population. Frequently, the population comprises a dialysis population made up of ESRD and pre-ESRD patients. These ranges, as further provided in Example 2, were generated from laboratory analysis of a large population of treated ESRD patients and published bone turnover data from non treated patients applied thereto and have basis therein. In one aspect, target ranges of the present invention include PTH agonist/antagonist, PTH agonist, or PTH antagonist ranges for a variety of persons without regard to limiting factors such as gender, race, age, etc. Because bone turnover rates may vary between selected individuals depending on such limiting factors, the normal PTH range for persons in one group (e.g., an adult) may not be the same as the normal PTH range for other persons in a different group (e.g., a child).

Any one discrete group may have a particular PTH-related (i.e., PTH agonist/antagonist ratio, PTH agonist level, PTH antagonist level) range. A different discrete group may have a different PTH-related range. A combination of one or more groups, e.g., a person with characteristic of the one or more groups, may yield an entirely different PTH-related range. Accordingly, a variety of combinations of discrete groups are possible and the normal PTH ranges for these groups may vary accordingly. In one aspect, the present invention contemplates the use of demographic data as providing an indication of an appropriate “normal” PTH-related range.

In general, demographic PTH-related data of the present invention must have been generated from an assay contemplated in the present invention. As the present invention incorporates the conversion of test results obtained from a variety of different days of performance of the same assays, demographic data may be “adjusted” according to the methods described herein to generate adjusted normal ranges for PTH-related levels. Adjusted normal ranges also preferably lie within the target ranges provided herein.

According to one aspect of the present invention, PTH target ranges have upper and lower limits as depicted in Table 1. Accordingly, target PTH agonist/antagonist ratio ranges generally lie between about 1.17 and about 3.15; target PTH agonist levels lie between about 83 pgm/ml and about 412 pgm/ml; target PTH antagonist levels lie between about 14 pgm/ml to about 91 pgm/ml. In a related aspect, the PTH agonist/antagonist ratio ranges, PTH agonist ranges, and PTH antagonist ranges may be adjusted ranges according to the present invention.

According to one aspect of the present invention, PTH agonist/antagonist ratio target ranges of the present invention incorporate a variety of “normal ranges” within their bounds, i.e., between 1.17 and 3.15. For purposes of illustration, the bottom of the normal range for PTH agonist/antagonist ratios may lie at or between about 1.17 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.14; and the top of the normal range may lie at or between about 1.18 to 1.25, 1.25 to 1.50, 1.50 to 1.75, 1.75 to 1.80, 1.80 to 1.95, 1.95 to 2.0, 2.0 to 2.25, 2.25 to 2.5, 2.5 to 2.75, 2.75 to 3.00, and 3.00 to 3.15. In a related aspect, normal PTH agonist/antagonist ranges of the present invention may be about one whole number unit in length, i.e., from 2 to 3. In a further related aspect, the PTH agonist/antagonist ratios may be adjusted PTH agonist ratios according to the present invention.

According to another aspect of the present invention, PTH agonist target ranges of the present invention incorporate a variety of “normal ranges” within their bounds, i.e., between about 83 and about 412 pgm/ml. For purposes of illustration, the bottom of the normal range for PTH agonist level may lie at about 83, 90, 100, 110, 120, 127, 130, 140, 150, 160, 170, 180, 190, 200, 210, 220, 230, 240, 250, 260, 270, 280, 290, 300, 310, 320, 330, 340, 350, 360, 370, 380, 390, 400, or about 410 pgm/ml; and the top of the normal range may lie at about 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, or about 412 pgm/ml. In a related aspect the PTH agonist levels may be adjusted PTH agonist levels according to the present invention.

According to another aspect of the present invention, PTH antagonist target ranges of the present invention incorporate a variety of “normal ranges” within their bounds, i.e., between about 14 and about 91 pgm/ml. For purposes of illustration, the bottom of the normal range for PTH antagonist level may lie at about 14, 15, 20, 25, 30, 35, 40, 45, 50, 55, 60, 65, 70, 75, 80, 85, or about 90 pgm/ml; and the top of the normal range may lie at about 20, 25, 30, 35, 40, 45, 50, 55, 60, 65, 70, 75, 80, 85, 90, or about 91 pgm/ml. In a related aspect the PTH antagonist levels may be adjusted PTH agonist levels according to the present invention.

PTH Assays

A. Scantibodies PTH Assays

An assay available from Scantibodies Laboratories is useful for the measurement of PTH agonist—Scantibodies CAP PTH assay or Scantibodies Whole PTH assay. An additional assay available from Scantibodies Laboratories is useful for the measurement of PTH agonist in addition to PTH antagonist—Scantibodies total intact PTH assay or Scantibodies intact PTH assay. The assays contemplated by Scantibodies Laboratories have generated ratios that have been validated as accurate through bone biopsies of patients. As provided above, the measurement of both PTH agonist and total PTH level provides an accurate measurement of the PTH antagonist level in a sample.

B. Non-Scantibodies Intact PTH Assays Non-Scantibodies 3rd Generation Assays

Non-Scantibodies intact PTH assays and non-Scantibodies 3rd Generation assays generally refer to assays not directly available from Scantibodies Laboratories, Inc., Santee, Calif. For example, a non-Scantibodies 3rd Generation assay directed to the measurement of PTH agonist, as defined herein, may be selected from a Nichols Institute Diagnostics Advantage Bio-Intact PTH assay or an Immutopics Human Bioactive Intact PTH assay. A non-Scantibodies intact PTH assay directed to the measurement of total PTH may be selected from a Nichols Institute Diagnostics Allegro Intact PTH Assay, Nichols Institute Diagnostics Advantage Intact PTH Assay, or an Immutopics Human Intact PTH assay. See, e.g., Slatopolsky E, et al., Kidney Intl. 2000; 58:753-761 (demonstrating that both that the Nichols Allegro intact PTH IRMA test measures both the 1-84 PTH and the 7-84 PTH and that the 7-84 PTH is an antagonist of the 1-84 PTH). Nichols Institute Diagnostics PTH-related assays referred to herein are generally available from Nichols Institute Diagnostics, San Clemente, Calif. Immutopics PTH-related assays referred to herein are generally available from Immutopics, Inc., San Clemente, Calif.

Other features and advantages of the invention will be apparent from the following detailed description, and from the claims.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

Table 2 below depicts a comparison of Scantibodies (SCL) assays and Nichols assays. As indicated in the table, treatment decisions vary widely between the PTH agonist/PTH antagonist ratios obtained for the same samples by the Scantibodies and Nichols assays. For 88% (15 out of 17) of the dialysis patients in this study an opposite Vitamin D treatment adjustment would have been made based on the results of the different assays.

TABLE 2

| | SCL iPTH | SCL 1-84 PTH | SCL ratio | Nichols iPTH | Nichols 1-84 PTH | Nichols Ratio |
|---|---|---|---|---|---|---|
| Patient | pgm/ml | pgm/ml | (SCL Vitamin D decision) | pgm/ml | pgm/ml | (Nichols Vitamin D decision) |
| 1 | 116 | 71.6 | 1.6 (increase) | 268 | 127 | 0.9 (stop) |
| 2 | 288.5 | 177 | 1.6 (increase) | 588 | 200 | 0.5 (stop) |
| 3 | 142.4 | 101.4 | 2.5 (increase) | 146 | 70 | 0.9 (stop) |
| 4 | 210.2 | 167.3 | 3.9 (increase) | 280 | 139 | 1.0 (stop) |
| 5 | 1018.8 | 719.4 | 2.4 (increase) | 1212 | 600 | 1.0 (stop) |
| 6 | 74.5 | 45.1 | 1.5 (increase) | 138 | 57 | 0.7 (stop) |
| 7 | 101.4 | 59.2 | 1.4 (no change) | 168 | 76 | 0.8 (stop) |
| 8 | 184.2 | 140.5 | 3.2 (increase) | 397 | 221 | 1.3 (no change) |
| 9 | 289.9 | 175.1 | 1.5 (no change) | 444 | 168 | 0.6 (stop) |
| 10 | 181.2 | 96.1 | 1.1 (no change) | 234 | 94 | 0.7 (stop) |
| 11 | 30.6 | 21.1 | 2.2 (increase) | 66 | 29 | 0.8 (stop) |
| 12 | 91.7 | 59.4 | 1.8 (increase) | 166 | 76 | 0.8 (stop) |
| 13 | 115.2 | 63 | 1.2 (no change) | 183 | 85 | 0.9 (stop) |
| 14 | 272.5 | 195.3 | 2.5 (increase) | 243 | 108 | 0.8 (stop) |
| 15 | 92.6 | 39.9 | 0.8 (stop) | 275 | 114 | 0.7 (stop) |
| 16 | 435.8 | 300.5 | 2.2 (increase) | 355 | 144 | 0.7 (stop) |
| 17 | 83.2 | 32.6 | 0.6 (stop) | 227 | 82 | 0.6 (stop) |

### Example 2

A population of 2185 dialysis patients was selected for routine PTH level analysis. To generate a target range of bone turnover rates for a variety of discrete patient populations (as described above) receiving kidney dialysis, several factors were taken into account. According to Monier-Faugere, et al. Kidney Int'l (2001) 60:1460-68, 52% of patients receiving dialysis treatment have adynamic bone disease. This percentage was based on patient populations prior to receiving Vitamin D therapy. Therefore, subsequent to, or concurrent with, Vitamin D treatment, a similar selection of patients would have a higher percentage incidence of adynamic bone disease due to the PTH suppressive effect of Vitamin D therapy. And, of note, the majority of dialysis patients are subject to Vitamin D therapy. In addition, in Monier-Faugere et al., the patient selection was comprised of 65% peritoneal dialysis patients. Notably, peritoneal dialysis patients generally comprise about 10% in a normal population. And, peritoneal dialysis patients generally have a much higher incidence of adynamic low bone turnover disease because the dialysis solution that is pumped into the peritoneum of these patients typically has a higher calcium concentration than the dialysate bath calcium concentration solution used for hemodialysis patients and the peritoneum solution remains in the peritoneum for hours during which the exchange takes place. See Taylor P M, Semin. Dial. July-August (2002);15(4):250-8; Richards P J, et al., Clin. Nephrol. February (1999);51(2):126-7; Stafford-Johnson D B, et al., J. Comput. Assist. Tomogr. March-April (1998);22(2):295-9; Kuriyama S, et al., Blood Purif. (1998);16(1):43-8.

Based on the foregoing, a target range was generated based on the baseline 52% indicated by Monier-Faugere et al. This range incorporated a plus 36% and minus 36% from the mid-point 52% mark to incorporate a reasonable range of expected patients having a variety of medical histories and bone turnover rates. The range generated, as correlated with the patient population analyzed, allowed for an upper and lower cutoff, for example, a target range between 16% and 88% of the patient population described above. Without being bound by theory, as described above, subsets of discrete patient populations may fall within this target range.

The target range of bone turnover rates was then applied to the PTH agonist versus PTH antagonist ratio as determined by Scantibodies Laboratory Whole PTH Assay, Scantibodies Laboratory CAP Assay, Scantibodies Laboratory Intact PTH Assay, and Scantibodies Laboratory Total Intact PTH Assay. The PTH agonist/antagonist ratios were calculated for the 2185 member patient population. The patient results were then listed from 1 to 2185 in terms of increasing PTH agonist/antagonist ratios. A target PTH agonist/antagonist ratio range was then determined for the patient population between the 16% mark (16% from the lowest ratio) and the 88% mark (88% from the lowest ratio). In addition, a mid-point PTH agonist/antagonist ratio was determined at the 52% mark (52% from the lowest ratio).

Patient data at the 16% mark corresponded to a PTH agonist/antagonist ratio of about 1.17, patient data at the 88% mark corresponded to a ratio of about 3.15, and patient data at the 52% mark corresponded to a ratio of about 1.8. Therefore, without being bound by theory, the resulting predicted target range of incidence of adynamic low bone turnover disease in the tested patient population, as characterized in terms of PTH agonist/antagonist ratios, was between 1.17 and 3.15 with a mid-point of 1.8.

### Example 3

Target ranges for PTH agonist levels were then generated based on the percentages and ranges generated in Example 2 (i.e., a mid-point of 52%, as indicated by Monier-Faugere et al., plus 36% and minus 36%). The range generated, as correlated with the patient population analyzed, allowed for an upper and lower cutoff including a target range between 16% and 88% of the patient population.

A population of 2237 dialysis patients was selected for routine PTH level analysis. The PTH agonist levels were then calculated for the 2237 member patient population. The patient results were then listed by increasing PTH agonist levels. A target PTH agonist level range was then determined for the patient population between the 16% mark (16% from the lowest PTH agonist level) and the 88% mark (88% from the lowest PTH agonist level). In addition, a mid-point PTH agonist level was determined at the 52% mark (52% from the lowest PTH agonist level). Patient data at the 16% mark corresponded to a PTH agonist level of about 83 pgm/ml, patient data at the 88% mark corresponded to a PTH agonist level of about 412 pgm/ml, and patient data at the 52% mark corresponded to a PTH agonist level of about 127 pgm/ml. Therefore, without being bound by theory, the resulting predicted target range of incidence of adynamic low bone turnover disease in the tested patient population, as characterized in terms of PTH agonist levels, was between about 83 pgm/ml and about 412 pgm/ml with a mid-point of about 127 pgm/ml. These ranges are represented above in Table 1.

### Example 4

Target ranges for PTH antagonist levels were then generated based on the percentages and ranges generated in Example 2 (i.e., a baseline of 52%, as indicated by Monier-Faugere et al., plus 36% and minus 36%). The range generated, as correlated with the patient population analyzed, allowed for an upper and lower cutoff including a target range between 16% and 88% of the patient population.

A population of 2187 dialysis patients was selected for routine PTH level analysis. The PTH antagonist levels were then calculated for the 2187 member patient population. The patient results were then listed by decreasing PTH antagonist levels. A target PTH antagonist level range was then determined for the patient population between the 16% mark (16% from the highest PTH antagonist level) and the 88% mark (88% from the highest PTH antagonist level). In addition, a mid-point PTH antagonist level was determined at the 52% mark (52% from the highest PTH antagonist level). Patient data at the 16% mark corresponded to a PTH antagonist level of about 91 pgm/ml, patient data at the 88% mark corresponded to a PTH antagonist level of about 14 pgm/ml, and patient data at the 52% mark corresponded to a PTH antagonist level of about 63 pgm/ml. Therefore, without being bound by theory, the resulting predicted target range of incidence of adynamic low bone turnover disease in the tested patient population, as characterized in terms of PTH antagonist levels, was between about 14 pgm/ml and about 91 pgm/ml with a mid-point of about 63 pgm/ml. These ranges are represented above in Table 1.

### Example 5

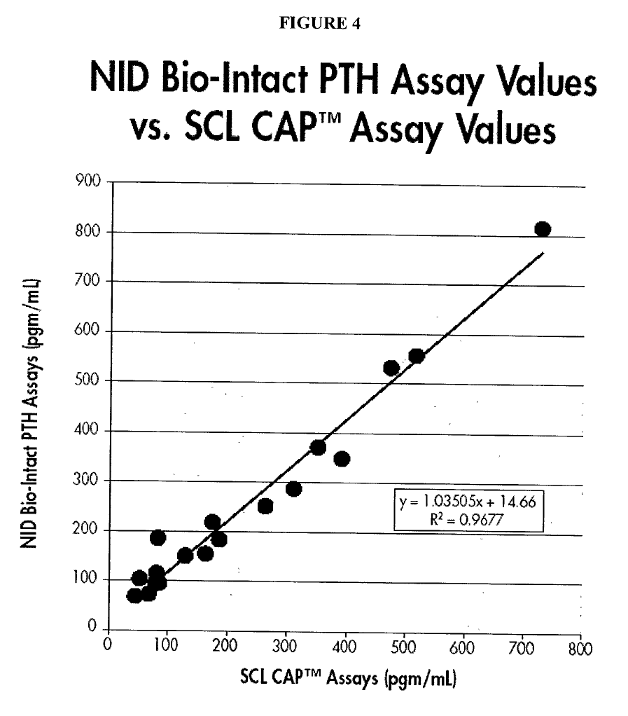

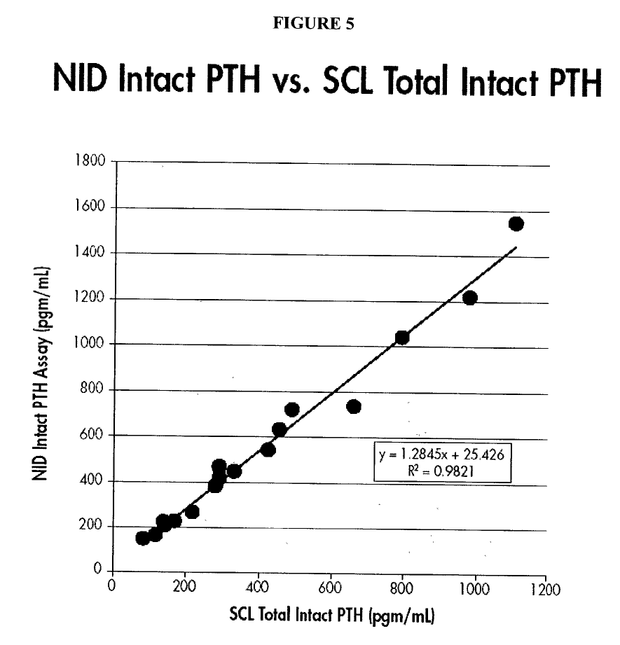

As presented in FIGS. 4-6, an analysis was undertaken to compare analogous results from different PTH assays to determine whether a correlation existed. As indicated in Table 3, several samples were subjected to PTH assays available from Scantibodies Laboratories and Nichols Institute Diagnostics. The values obtained for PTH agonist and total PTH yielded a significant correlation between these assays (R²=0.9677 for PTH agonist; and R²=0.9821 for total PTH) (see FIGS. 4 and 5).

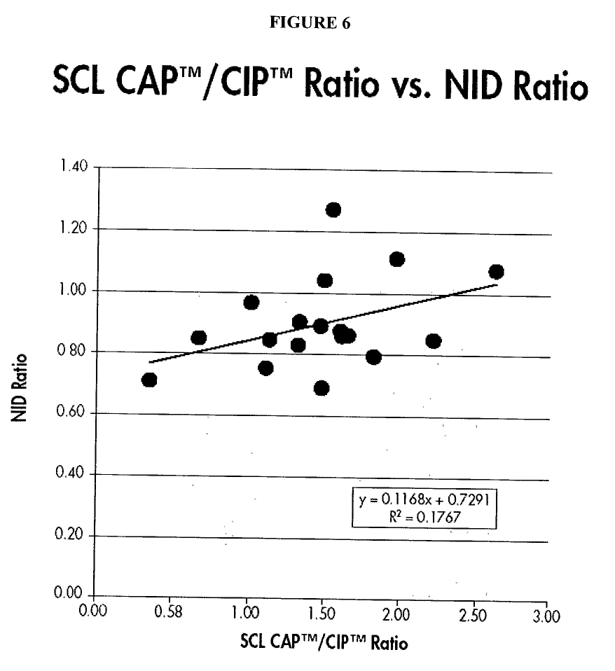

PTH agonist/antagonist ratios were then obtained through the use of the Scantibodies (Scantibodies total intact PTH assay and Scantibodies CAP assay) and Nichols (Nichols intact PTH assay and Nichols BioIntact PTH assay) assays. However, as indicated in Table 3 and FIG. 6, a direct correlation between the ratios obtained by these different assays was not obtained (R²=0.1767).

Based on the foregoing it was discovered that the values obtained by the different assays for total PTH and PTH agonist could be converted to the values obtained by the other analogous assay. A conversion or adjustment of this type was made possible through the generation of bias factors between the analogous assays for total PTH and PTH agonist levels. The adjusted values obtained for total PTH and PTH agonist are useful for generating adjusted PTH antagonist levels and adjusted PTH agonist/antagonist ratios.

Example conversions/adjustments of PTH values obtained through the use of Nichols assays (Nichols Intact and Nichols Bio-Intact) to corresponding values obtained through the use of Scantibodies assays (Scantibodies Total Intact and Scantibodies CAP) are presented in Table 4. As presented therein, bias values are calculated and used to adjust the total PTH and PTH agonist levels obtained through the use of the Nichols assays. Adjusted Nichols PTH agonist/antagonist ratios are then determined through the use of the adjusted total PTH and PTH agonist levels.

TABLE 3

| | | | | 2nd Generation | | 3rd Generation | | | |
|---|---|---|---|---|---|---|---|---|---|
| | | | Vitamin D | Intact PTH pgm/mL | | 1-84 PTH pgm/mL | | 1-84/7-84 PTH ratio | |
| | Calcium* | Phosphate | (Zemplar ®)^{#} | Scantibodies | Nichols | Scantibodies | Nichols | Scantibodies | Nichols |
| Patient | mg/dl | mg/dl | μgm | Total Intact | Intact | CAP | Bio-Intact | CAP/CIP ratio | Ratio |
| 1 | 8.7 | 6.0 | up to 6 | 423 | 540 | 263 | 250 | 1.6 | 0.86 |
| 2 | 9.5 | 7.4 | 4 | 288 | 464 | 177 | 217 | 1.6 | 0.88 |
| 3** | 10.8 | 10.0 | down to 14 | 331 | 446 | 83 | 185 | 0.3 | 0.71 |
| 4 | 9.2 | 7.0 | 8 | 141 | 205 | 80 | 93 | 1.3 | 0.83 |
| 5*** | 8.8 | 8.0 | 8 | 661 | 736 | 392 | 347 | 1.5 | 0.89 |
| 6 | 7.9 | 8.9 | start at 8 | 1101 | 1546 | 729 | 815 | 2.0 | 1.11 |
| 7 | 9.8 | 6.0 | down to 8 | 136 | 224 | 54 | 103 | 0.7 | 0.85 |
| 8 | 9.3 | 8.8 | 4 | 453 | 628 | 312 | 288 | 2.2 | 0.85 |
| 9 | 9.5 | 10.0 | 8 | 794 | 1042 | 475 | 531 | 1.5 | 1.04 |
| 10 | 9.7 | 7.5 | 4 | 79 | 141 | 45 | 67 | 1.3 | 0.91 |
| 11 | 8.8 | 8.4 | 8 | 216 | 268 | 131 | 150 | 1.5 | 1.27 |
| 12 | 9.5 | 9.8 | 6 | 277 | 379 | 165 | 155 | 1.5 | 0.69 |
| 13 | 8.9 | 5.9 | 2 | 290 | 413 | 187 | 183 | 1.8 | 0.80 |
| 14 | 8.4 | 6.6 | 4 | 164 | 232 | 82 | 114 | 1.0 | 0.97 |
| 15*** | 9.9 | 6.5 | down to 4 | 112 | 158 | 69 | 73 | 1.6 | 0.86 |
| 16 | 10.1 | 8.3 | 8 | 164 | 221 | 86 | 95 | 1.1 | 0.75 |
| 17 | 9.4 | 4.9 | hold | 978 | 1216 | 518 | 557 | 1.1 | 0.85 |
| 18 | 5.9 | 7.1 | up to 8 | 489 | 715 | 354 | 371 | 2.6 | 1.08 |

**Patient had intact PTH of 1773 pgm/ml 11/01. Zemplar had been titrated up to 20 μgm at this time.
***Patients did not tolerate Renagel (diarrhea)
^{#}Zemplar ® is available from Abbott Laboratories

TABLE 4

| | | | | | | | | | | Adjusted |
|---|---|---|---|---|---|---|---|---|---|---|
| | | | | Adjusted | | | | Adjusted | | Nichols PTH |
| | | | | Nichols | | | | Nichols | | agonist/ |
| | Scantibodies | Nichols | Bias | total | Scantibodies | Nichols | Bias | PTH | Scantibodies | antagonist |
| Patient | Total Intact | Intact | Factor | PTH | CAP | Bio-Intact | Factor | agonist | CAP/CIP ratio | ratio |
| 1 | 423 | 540 | 0.783 | 423 | 263 | 250 | 1.052 | 263 | 1.6 | 1.6 |
| 2 | 288 | 464 | 0.621 | 288 | 177 | 217 | 0.81567 | 177 | 1.6 | 1.6 |
| 3** | 331 | 446 | 0.742 | 331 | 83 | 185 | 0.44865 | 83 | 0.3 | 0.3 |
| 4 | 141 | 205 | 0.6878 | 141 | 80 | 93 | 0.86022 | 80 | 1.3 | 1.3 |
| 5*** | 661 | 736 | 0.8981 | 661 | 392 | 347 | 1.1297 | 392 | 1.5 | 1.5 |
| 6 | 1101 | 1546 | 0.71216 | 1101 | 729 | 815 | 0.89448 | 729 | 2.0 | 2.0 |
| 7 | 136 | 224 | 0.60714 | 136 | 54 | 103 | 0.52427 | 54 | 0.7 | 0.7 |
| 8 | 453 | 628 | 0.72134 | 453 | 312 | 288 | 1.0833 | 312 | 2.2 | 2.2 |
| 9 | 794 | 1042 | 0.762 | 794 | 475 | 531 | 0.89454 | 475 | 1.5 | 1.5 |
| 10 | 79 | 141 | 0.56 | 79 | 45 | 67 | 0.67164 | 45 | 1.3 | 1.3 |
| 11 | 216 | 268 | 0.80597 | 216 | 131 | 150 | 0.87333 | 131 | 1.5 | 1.5 |
| 12 | 277 | 379 | 0.73087 | 277 | 165 | 155 | 1.0645 | 165 | 1.5 | 1.5 |
| 13 | 290 | 413 | 0.70218 | 290 | 187 | 183 | 1.0219 | 187 | 1.8 | 1.8 |
| 14 | 164 | 232 | 0.7069 | 164 | 82 | 114 | 0.7193 | 82 | 1.0 | 1.0 |
| 15*** | 112 | 158 | 0.70886 | 112 | 69 | 73 | 0.94521 | 69 | 1.6 | 1.6 |
| 16 | 164 | 221 | 0.74208 | 164 | 86 | 95 | 0.90526 | 86 | 1.1 | 1.1 |
| 17 | 978 | 1216 | 0.80428 | 978 | 518 | 557 | 0.93 | 518 | 1.1 | 1.1 |
| 18 | 489 | 715 | 0.68392 | 489 | 354 | 371 | 0.95418 | 354 | 2.6 | 2.6 |

**Patient had intact PTH of 1773 pgm/ml 11/01. Zemplar had been titrated up to 20 μgm at this time.
***Patients did not tolerate Renagel (diarrhea)

### Example 6

In a separate patient study it was discovered that a correlation existed between PTH antagonist levels and phosphate levels in a person. It was further discovered that the administration of Vitamin D (e.g., Hectoral®—available from Bone Care International, Inc.) to a patient could influence both the phosphate and PTH antagonist levels. A high dose of Vitamin D increases the concentration of PTH antagonist versus PTH agonist; conversely a lower dose of Vitamin D correlates with a higher ratio of PTH agonist versus PTH antagonist. The varying concentration of PTH antagonist also affected similar results in the phosphate concentration in the patient. Table 5 below summarizes these discoveries.

TABLE 5

| Intact | | | | | PTH | PTH | PTH |
|---|---|---|---|---|---|---|---|
| PTH | Ca | PO₄ | | Hectoral ® | agonist | antagonist | agonist/ |
| pgm/ml | mg/dl | mg/dl | Ca × PO₄ | ugm/dose | pgm/ml | pgm/ml | antagonist |
| 3559 | 8.3 | 7.0 | 58 | 4 | 1735 | 1824 | .095 |
| 1679 | 8.1 | 5.7 | 46 | 2.5 | 1462 | 217 | 6.74 |

The table above presents results from the same patient, subjected to a high dose of Vitamin D (top) and low dose of Vitamin D (bottom). The significance of the above findings is important because it is known that soft tissue calcification begins at a (Ca)×(PO₄) level above about 55 mg²/ml² in a patient. See, e.g., Block G A, Clin. Nephrol. October (2000);54(4):318-24. And, there are serious consequences to a patient from the mismanagement of calcium levels by either direct or indirect PTH suppression therapy. For example, soft tissue calcification has led to a five to fifteen times greater incidence of myocardial infarction among end stage renal dialysis patients compared to age matched diabetes patients. Therefore, monitoring of PTH antagonist levels, especially in relation to PTH agonist levels is important to avoid adverse consequences of high phosphate/PTH antagonist levels. In addition, the control of the PTH agonist/antagonist ratio in a person exhibits an effect on the control of the phosphate levels in a person. For example, without being bound by theory, an increase in the PTH agonist/antagonist ratio in a person results in a decrease in the person's blood phosphate level, and vice-versa.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

We claim:

1. A method for assessing a person's bone turnover rate comprising:
a) obtaining a sample from a person to be tested;
b) determining the level of a parathyroid hormone (PTH) agonist and a PTH antagonist in the sample;
c) obtaining a ratio of the PTH agonist versus the PTH antagonist for the person; and
d) comparing the ratio obtained in step c) to a list of probabilities for predicting adynamic low bone turnover disease expressed as a percentage for accurate prediction of an adynamic low bone turnover disease, the probabilities being in a relationship to PTH agonist/antagonist ratios based on a dialysis patient population, wherein the population has a clinically significant risk of an adynamic low bone turnover disease below a normal PTH agonist/antagonist ratio range within a target ratio range of between about 1.17 to about 3.15, and wherein the person is determined as having an adynamic low bone turnover disease if the ratio of step c) is below the normal range within the target ratio range.

2. The method of claim 1, wherein a person has a clinically significant risk of an adynamic low bone turnover disease at a ratio of less than about 1.8.

3. The method of claim 1, wherein a person has a clinically significant risk of an adynamic low bone turnover disease at a ratio of less than about 1.17.

4. The method of claim 1, wherein a person has a high bone turnover rate at a ratio of above about 3.15.

5. The method of claim 1, wherein a person has a normal or high bone turnover rate at a ratio of above about 1.8.

6. The method of claim 1, wherein a person has a normal bone turnover rate at a ratio of between about 1.17 and about 3.15.

7. The method of claim 1, wherein the ratio of PTH agonist versus PTH antagonist is determined using an immunoassay.

8. The method of claim 1, wherein the sample is selected from the group consisting of a serum, a plasma and a blood sample.

9. The method of claim 1, wherein the person is a patient receiving routine dialysis treatments.

10. The method of claim 1, wherein the person is a pre-end stage renal dialysis patient.

11. The method of claim 9, wherein the person is an end-stage renal disease (ESRD) patient.

12. The method of claim 9, wherein the person is a patient with impaired renal function at a pre end-stage renal disease (ESRD) status with a glomerular filtration rate (GFR) of less than 60 ml/ml.

13. The method of claim 1, wherein the person is an osteoporosis patient.

14. The method of claim 1, wherein the person is a patient with bone disease.

15. The method of claim 1, wherein the population comprises an ESRD patient population.

16. The method of claim 15, wherein about 52% of the ESRD population has a clinically significant risk of an adynamic low bone turnover disease at a ratio below a normal range within the target ratio range of between about 1.17 to about 3.15.

17. The method of claim 15, wherein the majority of the ESRD patient population has been subjected to Vitamin D, Vitamin D analog, calcimimetic or calcium supplement treatment.

18. The method of claim 1, wherein the PTH agonist comprises a contiguous portion of human PTH having an amino acid sequence set forth in SEQ ID NO:1 (PTH₁₋₈₄), and the PTH agonist has the following characteristics:
a) the N-terminal amino acid residue of the PTH agonist starts at position 1 of the PTH₁₋₈₄; and
b) the C-terminal amino acid residue of the PTH agonist ends at any position spanning position 34 through position 84 of the PTH₁₋₈₄.

19. The method of claim 1, wherein the PTH agonist is a peptide having an amino acid sequence of human PTH₁₋₈₄.

20. The method of claim 1, wherein the PTH antagonist comprises a contiguous portion of human PTH having an amino acid sequence set forth in SEQ ID NO:1 (PTH₁₋₈₄), and the PTH antagonist has the following characteristics:
a) the N-terminal amino acid residue of the PTH antagonist starts at any position spanning position 2 through position 33 of the PTH₁₋₈₄;
b) the C-terminal amino acid residue of the PTH antagonist ends at any position spanning position 35 through position 84 of the PTH₁₋₈₄; and
c) the PTH antagonist has a minimal length of three amino acid residues.

21. The method of claim 1, wherein the PTH antagonist is a peptide having an amino acid sequence of human PTH₇₋₈₄.

22. The method of claim 1, wherein the parathyroid hormone agonist level is determined using an antibody that distinguishes a PTH agonist from a PTH antagonist.

23. The method of claim 21, wherein the antibody is an antibody or an antibody fragment specific for the PTH peptide SER-VAL-SER-GLU-ILE-GLN (SEQ ID NO:2).

24. The method of claim 21, wherein the antibody is a anti-(1-6) PTH antibody, anti-(1-4) PTH antibody, anti-(1-9) PTH antibody, anti-(1-11) PTH antibody, anti-(1-12) PTH antibody, or a combination thereof.

25. The method of claim 1, wherein the PTH antagonist level is determined by determining a total PTH level and determining a PTH agonist level followed by subtracting the PTH agonist level from the total PTH level.

26. The method of claim 1, wherein the PTH agonist and PTH antagonist levels and the corresponding ratio are calculated using a Scantibodies Laboratory Whole PTH Assay, Scantibodies Laboratory CAP Assay, Scantibodies Laboratory Intact PTH Assay, Scantibodies Laboratory Total Intact PTH Assay or a combination thereof.

27. The method of claim 1, wherein the PTH agonist are determined and PTH antagonist levels and the corresponding ratio are calculated using a Nichols Advantage Intact PTH Assay, a Nichols Advantage BioIntact PTH Assay, a Nichols Allegro Intact PTH Assay or a combination thereof.

28. A method of guiding therapy for a person suspected of having an adynamic low bone turnover disease comprising determining the ratio of claim 1 and determining therapy based thereon, wherein:
a) at a ratio below the normal ratio range, therapy to increase the bone turnover rate in the person is started or increased, or therapy to decrease the bone turnover rate in the person is halted or decreased;
b) at a ratio above the normal ratio range, therapy to decrease the bone turnover rate in the person is started or increased, or therapy to increase the bone turnover rate in the person is halted or decreased; and
c) at a ratio within the normal ratio range, no bone turnover-related therapy is begun or altered.

29. The method of claim 28, wherein the therapy to decrease the bone turnover rate in the person comprises Vitamin D, Vitamin D analog, calcimimetic, calcium supplement therapy, PTH antagonist administration or a combination thereof.

30. The method of claim 28, wherein the therapy to increase the bone turnover rate in the person comprises administering PTH agonist, phosphate, calcililetic, EDTA, calcium binding agent, or stimulating PTH production or a combination thereof.

31. A method for assessing a person's bone turnover rate comprising:
a) obtaining a sample from a person to be tested;
b) determining and comparing a total PTH level by two assays to generate a total PTH bias factor, the assays comprising
(1) a Scantibodies Laboratory Total Intact PTH Assay or a Scantibodies Laboratory Intact PTH Assay, or a combination thereof, and
(2) a non-Scantibodies Laboratory intact PTH assay;
c) determining and comparing a PTH agonist level by two PTH assays to generate a PTH agonist bias factor, the assays comprising
(1) a Scantibodies Laboratory Whole PTH Assay or a Scantibodies Laboratory CAP Assay, or a combination thereof, and
(2) a non-Scantibodies Laboratory 3^{rd} generation PTH assay;
d) adjusting the total PTH level determined by the non-Scantibodies Laboratory intact PTH assay, whereby the total PTH bias factor is multiplied by the total PTH level determined by the non-Scantibodies Laboratory intact PTH assay to obtain an adjusted total PTH level;
e) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level;
f) obtaining an adjusted PTH antagonist level by subtracting the adjusted PTH agonist level from the adjusted total PTH level;
g) obtaining an adjusted ratio of the adjusted PTH agonist versus the adjusted PTH antagonist; and
h) comparing the adjusted ratio to a list of probabilities expressed as a percentage for an adynamic low bone turnover disease, the probabilities being in a relationship to PTH agonist/antagonist ratios based on a dialysis patient population, wherein the population has a clinically significant risk of an adynamic low bone turnover disease below a normal PTH agonist/antagonist ratio range within a target ratio range of between about 1.17 to about 3.15, and wherein the person is determined as having an adynamic low bone turnover disease if the adjusted ratio of step g) is below the normal range within the target ratio range.

32. The method of claim 31, wherein the non-Scantibodies Laboratory 3rd generation PTH assay or the non-Scantibodies Laboratory intact PTH assay is selected from the group consisting of Nichols Institute Diagnostics Allegro Intact PTH Assay, Nichols Institute Diagnostics Advantage Bio-Intact PTH Assay, Immutopics Human BioActive Intact PTH assay, and Immutopics Human Intact PTH assay.

33. The method of claim 31, wherein the non-Scantibodies Laboratory 3rd generation PTH assay or the non-Scantibodies PTH assay is the same or different assay between steps b) and c).

34. The method of claim 31, wherein the total PTH bias factor is obtained by dividing the total PTH value obtained through the practice of the step b)(2) by the corresponding total PTH value obtained through the practice of the assay of step b)(1); and
wherein the PTH agonist bias factor is obtained by dividing the PTH agonist value obtained through the practice of the assay of step c)(2) by the corresponding PTH agonist value obtained through the practice of step c)(1).

35. The method of claim 31, wherein a person has a clinically significant risk of an adynamic low bone turnover disease at an adjusted ratio of less than about 1.8.

36. The method of claim 31, wherein a person has a clinically significant risk of an adynamic low bone turnover disease at an adjusted ratio of less than about 1.17.

37. A method of guiding therapy for persons suspected of having an adynamic low bone turnover disease comprising determining a ratio of claim 31 and determining Vitamin D, Vitamin D analog, calcimimetic or calcium supplement therapy based thereon.

38. The method of claim 31, wherein a person has a high bone turnover rate at a ratio of above about 3.15.

39. The method of claim 31, wherein a person has a normal or high bone turnover rate at a ratio of above about 1.8.

40. The method of claim 31, wherein a person has a normal bone turnover rate at a ratio of between about 1.17 and about 3.15.

41. The method of claim 31, wherein the person is a patient receiving routine dialysis treatments.

42. The method of claim 31, wherein the person is a pre-end stage renal dialysis patient.

43. The method of claim 41, wherein the person is an end-stage renal disease (ESRD) patient.

44. The method of claim 41, wherein the person is a patient with impaired renal function at a pre end-stage renal disease (ESRD) status with a glomerular filtration rate (GFR) of less than 60 ml/ml.

45. The method of claim 31, wherein the person is an osteoporosis patient.

46. The method of claim 31, wherein the person is a patient with bone disease.

47. A method of guiding therapy for a person suspected of having an adynamic low bone turnover disease comprising determining the ratio of claim 31 and determining therapy based thereon, wherein:
a) at an adjusted ratio below the normal ratio range, therapy to increase the bone turnover rate in the person is started or increased, or therapy to decrease the bone turnover rate in the person is halted or decreased;
b) at an adjusted ratio above the normal ratio range, therapy to decrease the bone turnover rate in the person is started or increased, or therapy to increase the bone turnover rate in the person is halted or decreased; and
c) at an adjusted ratio within the normal ratio range, no bone turnover-related therapy is begun or altered.

48. The method of claim 47, wherein the therapy to decrease the bone turnover rate in the person comprises Vitamin D, Vitamin D analog, calcimimetic, calcium supplement therapy, PTH antagonist administration or a combination thereof.

49. The method of claim 47, wherein the therapy to increase the bone turnover rate in the person comprises administering PTH agonist, phosphate, EDTA, calcium binding agent, calcililetic, stimulating PTH production or a combination thereof.

50. A method for assessing a person's bone turnover rate comprising:
a) obtaining a sample from a person to be tested;
b) determining the level of a parathyroid hormone (PTH) agonist; and
c) comparing the PTH agonist level to a list of probabilities for predicting adynamic low bone turnover disease expressed as a percentage for accurate prediction of an adynamic low bone turnover disease, the probabilities being in a relationship to PTH agonist levels based on a dialysis patient population, wherein the population has a clinically significant risk of an adynamic low bone turnover disease below a normal PTH agonist range within a target PTH agonist range of between about 83 pgm/ml to about 412 pgm/ml, and wherein the person is determined as having an adynamic low bone turnover disease if the PTH agonist level is below the normal range within the target range.

51. The method of claim 50, wherein the person has a clinically significant risk of an adynamic low bone turnover disease at a PTH agonist level of below about 127 pgm/ml.

52. The method of claim 50, wherein a person has a high bone turnover rate at a PTH agonist level above about 412 pgm/ml.

53. The method of claim 50, wherein a person has a normal or high bone turnover rate at a PTH agonist level above about 127 pgm/ml.

54. The method of claim 50, wherein a person has a normal bone turnover rate at a PTH agonist level between about 83 pgm/ml and about 412 pgm/ml.

55. A method of guiding therapy a person suspected of having an adynamic low bone turnover disease comprising determining the PTH agonist level of claim 50 and determining therapy based thereon, wherein:
a) at a PTH agonist level below the normal range, therapy to increase the bone turnover rate in the person is started or increased, or therapy to decrease the bone turnover rate in the person is halted or decreased;
b) at a PTH agonist level above the normal range, therapy to decrease the bone turnover rate in the person is started or increased, or therapy to increase the bone turnover rate in the person is halted or decreased; and
c) at a PTH agonist level within the normal range, no bone turnover-related therapy is begun or altered.

56. The method of claim 55, wherein the therapy to decrease the bone turnover rate in the person comprises Vitamin D, Vitamin D analog, calcimimetic, calcium supplement therapy, PTH antagonist administration or a combination thereof.

57. The method of claim 50, wherein the therapy to increase the bone turnover rate in the person comprises administering PTH agonist, phosphate, EDTA, calcium binding agent, calcililetic, stimulating PTH production or a combination thereof.

58. A method for assessing a person's bone turnover rate comprising:
a) obtaining a sample from a person to be tested;
b) determining the level of a parathyroid hormone (PTH) antagonist; and
c) comparing the PTH antagonist level to a list of probabilities for predicting adynamic low bone turnover disease expressed as a percentage for accurate prediction of an adynamic low bone turnover disease, the probabilities being in a relationship to PTH antagonist levels based on a dialysis patient population, wherein the population has a clinically significant risk of an adynamic low bone turnover disease above a normal PTH antagonist range within the target PTH antagonist range of between about 14 pgm/ml to about 91 pgm/ml, and wherein the person is determined as having an adynamic low bone turnover disease if the PTH antagonist level is above the normal range within the target range.

59. The method of claim 58, wherein the person has a clinically significant risk of an adynamic low bone turnover disease at a PTH antagonist level of above about 63 pgm/ml.

60. The method of claim 58, wherein a person has a high bone turnover rate at a PTH antagonist level below about 14 pgm/ml.

61. The method of claim 58, wherein a person has a normal or high bone turnover rate at a PTH antagonist level below about 63 pgm/ml.

62. The method of claim 58, wherein a person has a normal bone turnover rate at a PTH antagonist level between about 14 pgm/ml and about 91 pgm/ml.

63. A method of guiding therapy a person suspected of having an adynamic low bone turnover disease comprising determining the PTH antagonist level of claim 58 and determining therapy based thereon, wherein:
a) at a PTH antagonist level above the normal range, therapy to increase the bone turnover rate in the person is started or increased, or therapy to decrease the bone turnover rate in the person is halted or decreased;
b) at a PTH antagonist level below the normal range, therapy to decrease the bone turnover rate in the person is started or increased, or therapy to increase the bone turnover rate in the person is halted or decreased; and
c) at a PTH antagonist level within the normal range, no bone turnover-related therapy is begun or altered.

64. The method of claim 63, wherein the therapy to decrease the bone turnover rate in the person comprises Vitamin D, Vitamin D analog, calcimimetic, calcium supplement therapy, PTH antagonist administration or a combination thereof.

65. The method of claim 63, wherein the therapy to increase the bone turnover rate in the person comprises administering PTH agonist, phosphate, EDTA, calcium binding agent, calcililetic, stimulating PTH production or a combination thereof.

66. A method for assessing a person's bone turnover rate comprising:
a) obtaining a sample from a person to be tested;
b) determining and comparing a PTH agonist level by two PTH assays to generate a PTH agonist bias factor, the assays comprising
(1) a Scantibodies Laboratory Whole PTH Assay or a Scantibodies Laboratory CAP Assay, or a combination thereof, and
(2) a non-Scantibodies Laboratory 3^{rd} generation PTH assay;
c) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level; and
d) comparing the adjusted PTH agonist level to a list of probabilities for predicting adynamic low bone turnover disease expressed as a percentage for accurate prediction of an adynamic low bone turnover disease, the probabilities being in a relationship to PTH agonist levels based on a dialysis patient population, wherein the population has a clinically significant risk of an adynamic low bone turnover disease below a normal PTH agonist range within a target PTH agonist range of between about 83 pgm/ml to about 412 pgm/ml, and wherein the person is determined as having an adynamic low bone turnover disease if the adjusted PTH agonist level is below the normal range within the target range.

67. The method of claim 66, wherein the person has a clinically significant risk of an adynamic low bone turnover disease at an adjusted PTH agonist level of below about 127 pgm/ml.

68. The method of claim 66, wherein a person has a high bone turnover rate at an adjusted PTH agonist level above about 412 pgm/ml.

69. The method of claim 66, wherein a person has a normal or high bone turnover rate at an adjusted PTH agonist level above about 127 pgm/ml.

70. The method of claim 66, wherein a person has a normal bone turnover rate at an adjusted PTH agonist level between about 83 pgm/ml and about 412 pgm/ml.

71. A method of guiding therapy a person suspected of having an adynamic low bone turnover disease comprising determining the adjusted PTH agonist level of claim 66 and determining therapy based thereon, wherein:
a) at an adjusted PTH agonist level below the normal range, therapy to increase the bone turnover rate in the person is started or increased, or therapy to decrease the bone turnover rate in the person is halted or decreased;
b) at an adjusted PTH agonist level above the normal range, therapy to decrease the bone turnover rate in the person is started or increased, or therapy to increase the bone turnover rate in the person is halted or decreased; and
c) at an adjusted PTH agonist level within the normal range, no bone turnover-related therapy is begun or altered.

72. A method for assessing a person's bone turnover rate comprising:
a) obtaining a sample from a person to be tested;
b) determining and comparing a total PTH level by two assays to generate a total PTH bias factor, the assays comprising
(1) a Scantibodies Laboratory Total Intact PTH Assay or a Scantibodies Laboratory Intact PTH Assay, or a combination thereof, and
(2) a non-Scantibodies Laboratory intact PTH assay;
c) determining and comparing a PTH agonist level by two PTH assays to generate a PTH agonist bias factor, the assays comprising
(1) a Scantibodies Laboratory Whole PTH Assay or a Scantibodies Laboratory CAP Assay, or a combination thereof, and
(2) a non-Scantibodies Laboratory 3^{rd} generation PTH assay;
d) adjusting the total PTH level determined by the non-Scantibodies Laboratory intact PTH assay, whereby the total PTH bias factor is multiplied by the total PTH level determined by the non-Scantibodies Laboratory intact PTH assay to obtain an adjusted total PTH level;
e) adjusting the PTH agonist level determined by the non-Scantibodies Laboratory 3^{rd} generation PTH assay, whereby the PTH agonist bias factor is multiplied by the PTH agonist level determined by the non-Scantibodies Laboratory 3^{rd} generation PTH assay to obtain an adjusted PTH agonist level;
f) obtaining an adjusted PTH antagonist level by subtracting the adjusted PTH agonist level from the adjusted total PTH level; and
g) comparing the PTH antagonist level to a list of probabilities for predicting adynamic low bone turnover disease expressed as a percentage for accurate prediction of an adynamic low bone turnover disease, the probabilities being in a relationship to PTH antagonist levels based on a dialysis patient population, wherein the population has a clinically significant risk of an adynamic low bone turnover disease above a normal PTH antagonist range within a target PTH antagonist range of between about 14 pgm/ml to about 91 pgm/ml, and wherein the person is determined as having an adynamic low bone turnover disease if the PTH antagonist level is above the normal range within the target range.

73. The method of claim 72, wherein the person has a clinically significant risk of an adynamic low bone turnover disease at an adjusted PTH antagonist level of above about 63 pgm/ml.

74. The method of claim 72, wherein a person has a high bone turnover rate at an adjusted PTH antagonist level below about 14 pgm/ml.

75. The method of claim 72, wherein a person has a normal or high bone turnover rate at an adjusted PTH antagonist level below about 63 pgm/ml.

76. The method of claim 72, wherein a person has a normal bone turnover rate at an adjusted PTH antagonist level between about 14 pgm/ml and about 91 pgm/ml.

77. A method of guiding therapy a person suspected of having an adynamic low bone turnover disease comprising determining the adjusted PTH antagonist level of claim 61 and determining therapy based thereon, wherein:
a) at an adjusted PTH antagonist level above the normal range, therapy to increase the bone turnover rate in the person is started or increased, or therapy to decrease the bone turnover rate in the person is halted or decreased;
b) at an adjusted PTH antagonist level below the normal range, therapy to decrease the bone turnover rate in the person is started or increased, or therapy to increase the bone turnover rate in the person is halted or decreased; and
c) at an adjusted PTH antagonist level within the normal range, no bone turnover-related therapy is begun or altered.

78. A method for controlling the phosphate level in a person comprising:
a) obtaining a sample from a person to be tested;
b) determining the level of a parathyroid hormone (PTH) agonist and a PTH antagonist in the sample;
c) obtaining a ratio of the PTH agonist versus the PTH antagonist for the person; and
d) controlling the phosphate level in the patient based on an inverse correlation between the PTH agonist/antagonist ratio and the blood phosphate level, wherein when the PTH agonist/antagonist ratio increases, the blood phosphate level in the person decreases.

79. A method of controlling the phosphate level in a patient comprising determining the ratio between PTH agonist and PTH antagonist according to claim 78, and increasing the PTH antagonist level, wherein the phosphate level in the patient increases.

80. A method of controlling the phosphate level in a patient comprising determining the ratio between PTH agonist and PTH antagonist according to claim 78, and decreasing the PTH antagonist level, wherein the phosphate level in the patient decreases.

81. A method of controlling the phosphate level in a patient comprising determining the ratio between PTH agonist and PTH antagonist according to claim 78, and adjusting the PTH antagonist level to keep the product of the calcium and phosphate levels in the patient below about 55 mg²/ml².
